(19) United States
(12) Patent Application Publication
(10) Pub. No.: US 2026/0083873 A1
(43) Pub. Date: Mar. 26, 2026

(54) SUBSTRATE MATERIALS FOR EPIDERMAL USE ON SUBJECT, APPARATUS COMPRISING SAME, AND/OR METHODS OF MANUFACTURE THEREOF

(71) Applicant: City University of Hong Kong, Hong Kong (HK)

(72) Inventors: Yu FENG, Hong Kong (HK); Guanglie ZHANG, Shenzhen (CN); Wen Jung LI, Hong Kong (HK)

(73) Assignee: City University of Hong Kong, Hong Kong (HK)

(21) Appl. No.: 18/896,030

(22) Filed: Sep. 25, 2024

(51) Int. Cl. A61L 24/00; A61B 5/266; A61B 5/268

(52) U.S. Cl. CPC A61L 24/0078; A61B 5/266; A61B 5/268; A61B 2562/125

(57) ABSTRACT

One aspect of the present invention is concerned with a substrate material for epidermal use on a subject, e.g. human subject. The substrate material has a matrix ingredient of polyvinyl alcohol (PVA), a plasticizing agent of glycerol (gly), and a conductive material of graphite, carbon black, and water. Another aspect of the present invention is concerned with an electronic apparatus having such a substrate material and an electronic device for recording electrical signals received through the substrate material, wherein the electrical signals are indicative of one or more physiological or physical parameters of a subject on which the electronic apparatus is applied or one or more parameters of the surroundings to the subject. Yet a further aspect of the present invention is concerned with a method of making a substrate material.

### TECHNICAL FIELD OF THE INVENTION

The present invention is concerned with a substrate material, for epidermal use on subject, an apparatus or an electronic apparatus comprising same, and a method of manufacture thereof.

### BACKGROUND OF THE INVENTION

Epidermal electronic apparatus has garnered significant research attention due to its promising applications in wearable human-machine interfaces and intelligent healthcare sensing. However, their widespread use still faces various technical and commercial challenges due to complex manufacturing processes, high material costs, inadaptability to different skin surfaces, and/or inadequate reusability. For example, conventional epidermal electronics often have limited adhesion duration on the skin surface of a human subject. Once sufficient adhesion has been lost, the entire epidermal device needs to be replaced. Before a replacement can be arranged the subject would lose functionalities from the epidermal electronics. Replacement of the epidermal electronics is not only inconvenient, but also costly as well as discouraging from a subject compliance standpoint.

The present invention seeks to address the aforementioned shortcomings, or at least to provide alternatives to the public.

### SUMMARY OF THE INVENTION

According to a first aspect of the present invention, there is provided a substrate material comprising a matrix ingredient of polyvinyl alcohol (PVA), a plasticizing agent of glycerol (gly), and a conductive material of graphite, carbon black, and water.

Preferably, the substrate material may comprise a physical property enhancing agent of tannic acid (TA) for enhancing the mechanical performance of the substrate material. The physical property enhancing agent may be adapted to provide or improve crosslinking, adhesion, and/or flexibility in the substrate material. The substrate material may comprise a polymerization agent for improving cross-linking in the substrate material.

Advantageously, the substrate material may comprise 3.34-3.41 wt % of the polyvinyl alcohol (PVA) and 0.24-0.95 wt % of the glycerol (gly). The substrate material may have 1.865 to 3.22 wt % of the conductive material. The substrate material may comprise a polymerization agent for improving cross-linking in the substrate material, wherein the polymerization agent is selected from a group including borax pentahydrate, borax decahydrate, and glutaraldehyde, and wherein the substrate material preferably has 1-6 wt % of the polymerization agent and more preferably 1-4 wt % of the polymerization agent.

Suitably, the substrate material may comprise up to 0.71 wt % of the tannic acid (TA).

In an embodiment, the ratio of graphite to carbon black in the conductive material may be from 1:1 to 2.5:1.

In one embodiment, the weight ratio of the polyvinyl alcohol (PVA), glycerol (gly), tannic acid, and conductive material is 0.35:0.0625-0.225:0-0.15.

In another embodiment, the substrate material may have an adhesive strength of 2.42 kPa to 5.84 kPa, an elastic modulus of 2.67 kPa to 11.72 kPa, and a stretchability of up to 2600%.

In certain embodiments, the substrate material may assume the formation of a layer or a strip for attachment or engagement to the skin of a subject.

According to a second aspect of the present inventio, there is provided an electronic apparatus comprising a substrate material as described above and an electronic device for recording electrical signals received through the substrate material, wherein the electrical signals are indicative of one or more physiological or physical parameters of a subject on which the electronic apparatus is applied or one or more parameters of the surroundings to the subject. The parameters may be selected from the group including blood glucose level of the subject, electrocardiogram (ECG) of the subject, facial expression of the subject, temperature of the surroundings, humidity of the surroundings, motion and/or posture of the subject, wound healing status of the subject, and pressure or change of pressure at certain location of the subject.

According to a third aspect of the present invention, there is provided a method of making of a substrate material, comprising the steps of i) adding a predetermined amount of a matrix ingredient of polyvinyl alcohol (PVA) and a plasticizing agent of glycerol (gly) to deionized water to form a first solution, and mixing the solution for a period of time under a predetermined temperature, and ii) adding a conductive material of graphite and carbon black to the first solution and forming a second solution, and mixing the second solution for a period of time under a predetermined temperature to form a third solution.

Preferably, the method may comprise a step of also adding tannic acid (TA) to the first solution before adding the conductive material.

Advantageously, the method may comprise a step of adding a polymerization agent in the third solution, wherein the polymerization agent is selected from a group including borax pentahydrate and borax decahydrate or glutaraldehyde.

Suitably, the first solution may be stirred for 1-4 hours under 70-90° C. before adding the conductive material, and wherein the second solution is stirred for 5-20 mins under 70-90° C. before allowing the second solution to take form.

Advantageously, the method may comprise a step of shaping the formed substrate material into a predetermined configuration.

In an embodiment, the substrate material may have 3.34-3.41 wt % of the polyvinyl alcohol (PVA), 0.24-0.95 wt % of the glycerol (gly), 1.865 to 3.22 wt % of the conductive material, and up to 0.71 wt % of the tannic acid (TA), and the concentration of the polymerization agent is 1-6%.

### BRIEF DESCRIPTION OF THE DRAWINGS

Some embodiments of the present invention will now be explained, with reference to the accompanied drawings, in which:

FIG. 1Ai and FIG. 1Aii are optical images of an amoeba assuming different morphologies.

FIG. 1B is a schematic diagram illustrating the (full) biological reshapability of an amoeba from different morphologies.

FIG. 1C displays photographs illustrating the customizability and reshapability of a substrate material (also hereinafter referred to hydrogel material or E-slime) of the present invention when used on the skin surface of a human subject.

FIG. 1D displays photographs illustrating the application and ultra-deformability of the substrate material of the present invention by way of instant drawing on a skin surface of the human subject.

FIG. 1E displays photographs (top and middle) and a schematic diagram (bottom) illustrating the conformality of the substate material of the present invention when used on a skin surface of a human subject.

FIG. 1F schematically illustrates the stretchability and compressibility of the substrate material of the present invention.

FIG. 1G is a schematic diagram illustrating various applications of the substrate material of the present invention when used with electronics devices in electronic apparatus.

FIG. 1H displays graphs illustrating comparison between a substrate material (E-slime) of the present invention and conventional hydrogel materials (Silly Putty-like hydrogels) in terms of gauge factor versus conductivity (top), sensing range versus stretchability (middle), and self-healing time (bottom).

FIG. 2A is a schematic diagram illustrating an embodiment of a method of making a substrate material according to the present invention.

FIG. 2B is a schematic diagram illustrating the crosslinking structure of the substrate material made from the method of FIG. 2A.

FIG. 2C is a schematic diagram illustrating the bonding structure of the substrate material of FIG. 2B.

FIG. 2D is a schematic diagram illustrating the conductive mechanism of the substrate material of FIG. 2B.

FIG. 2E is a SEM image showing the conductive material disposed in the substate material of FIG. 2B.

FIG. 2F is a graph showing the XPS spectra of different substrate materials (hydrogels) of PVA, PVA/TA, PVA/gly, and PVA/gly/TA.

FIG. 2G is a graph showing the FTIR spectra of different prepared substrate materials (hydrogels).

FIG. 2H is a graph showing the viscosity of the different prepared materials (hydrogels) at room temperature.

FIG. 2I displays fluorescence images of the cultured C2C12 cells under different prepared materials (hydrogels).

FIG. 2J is a graph illustrating the cell viability in different prepared materials (hydrogels).

FIG. 3A is a graph illustrating the effect of glycerol content in PVA hydrogel materials on tensile stress strain.

FIG. 3B is a graph illustrating the effect of tannic acid mass content in PVA hydrogel materials on adhesive strength and elastic modulus.

FIG. 3C is a graph illustrating the effect of different conductive particle ratios of graphite-carbon black in PVA hydrogel materials on the elastic modulus and conductivity.

FIG. 3D illustrates the conductive mechanism of substrate material according to the present invention under shape changing.

FIG. 3E is a graph illustrating the effect of different graphite-to carbon black mass ratios in PVA hydrogel materials on the relative resistance variation as a function of strain.

FIG. 3F is a graph illustrating the effect of different graphite-to carbon black mass ratios in PVA hydrogel materials on the sheet resistance under different thickness.

FIG. 3G illustrates the strain resolution of two PVA hydrogels of the present invention when under 0% and 100% strain load.

FIG. 3H is a graph illustrating the relative resistance variation of different PVA hydrogels of the present invention with different thicknesses over time (room temperature).

FIG. 3I is a graph illustrating the resistance response of a PVA hydrogel of the present invention under different mechanical deformations on porcine skin.

FIG. 4A demonstrates the reshapability of an embodiment of a substrate material according to the present invention for customized deployment on skin.

FIG. 4B demonstrates the wide degree of shape changing of an embodiment of a substrate material according to the present invention.

FIG. 4C demonstrates the conductivity of reused substrate materials of the present invention in different shapes.

FIG. 4D shows the SEM images of substrate materials of the present invention under different conditions (initial and 500 cycles).

FIG. 4E is a graph illustrating the rheological characterizations of substrate materials of the present invention under different conditions (initial and 500 cycles).

FIG. 4F is a graph illustrating the elastic modulus of substrate materials of the present invention under different usage times.

FIG. 4G is a graph illustrating the conductivity of substrate materials of the present invention under different usage times/reshaping cycles.

FIG. 4H is a graph illustrating the adhesive strength of substrate materials of the present invention under different usage times.

FIG. 4I is a graph illustrating the resistance response of substrate materials of the present invention when changing different shapes on porcine skin.

FIG. 5A demonstrates the adhesive ability of substrates materials of the present invention on different substances.

FIG. 5B is a schematic illustration showing the principle of the bio-adhesive property of substrates materials of the present invention for skin.

FIG. 5C is a graph illustrating the adhesive ability of substrate materials of the present invention.

FIG. 5D demonstrates the self-healing ability of substrate materials of the present invention.

FIG. 5E demonstrates the molecular mechanism of self-healing property of substrate materials of the present invention.

FIG. 5F is a graph illustrating the resistance change of substrate materials of the present invention during cutting-healing cycles.

FIG. 5G is a graph illustrating the self-healing period of substrate materials of the present invention under different cutting distances.

FIG. 5H is a graph illustrating the conductivity of substrate materials of the present invention under different cutting-healing cycles.

FIG. 5I is a graph illustrating the elastic modulus of substrate materials of the present invention under different cutting-healing cycles.

FIG. 5J is a graph illustrating the relative resistance variation of substrate materials of the present invention under different cutting-healing cycles.

FIG. 6A is a schematic explanation providing an overview of sensing locations of a substrate material of the present invention placed on the hand of a subject.

FIG. 6B is a graph showing the resistance variation of the substrate material of FIG. 6A as a function of finger position angle.

FIG. 6C is a graph showing the real-time relative resistance variation of the substrate material under different finger bending angles (30°, 60°, and 90°).

FIG. 6D is a graph showing the real-time relative resistance variation of the substrate material under different thenar motion positions.

FIG. 6E is a graph showing the real-time relative resistance variation of the substrate material under different hand gestures.

FIG. 6F is a graph showing the real-time relative resistance variation of the substrate material when clicking the mouse.

FIG. 6G is a graph showing the real-time relative resistance variation of the substrate material when writing “hi.”

FIG. 6H is a graph showing the real-time relative resistance variation of the substrate material when writing the Chinese character “wo.”

FIG. 6I is a graph showing the real-time relative resistance variation (five channels) of the substrate material when clenching fist.

FIG. 6J is a graph showing the real-time relative resistance variation (five channels) of the substrate material under different hand gestures.

FIG. 7A is a schematic diagram providing an overview of sensing locations of the substrate material of the present invention applied on the body of a human subject.

FIG. 7B is a graph showing the real-time relative resistance variation under different facial expressions (frown and amaze).

FIG. 7C is a graph showing the real-time relative resistance variation under different mouth postures (smile, mouth open, and wide mouth open).

FIG. 7D is a graph showing the real-time relative resistance variation under different wrist bending angles (45° clockwise and counterclockwise).

FIG. 7E is a graph showing the real-time relative resistance variation under different elbow bending angles (30°, 45°, and 60°).

FIG. 7F is a graph showing the real-time relative resistance variation under different knee bending angles (45° clockwise/counterclockwise and 60° clockwise).

FIG. 7G is a graph showing the real-time relative resistance variation regarding ankle extension and flexion.

FIG. 7H is a graph showing the real-time relative resistance variation when swallowing and coughing.

FIG. 7I is a graph showing the real-time relative resistance variation when detecting pulse.

FIG. 8 displays the chemical structures of ingredients (polyvinyl alcohol, tannic acid, and glycerol) of a substrate material of the present invention.

FIG. 8 displays the chemical structures of ingredients (polyvinyl alcohol, tannic acid, and glycerol) of a substrate material of the present invention.

FIG. 9 displays the chemical structures of ingredients (graphite and carbon black granules) of a substrate material of the present invention.

FIG. 10A is a graph showing the high resolution XPS spectra of O, and FIG. 10B is a graph showing the high resolution XPS spectra of C.

FIG. 11 is a graph showing the gauge factor of a substrate material of the present invention with different ratios of graphite and carbon black in the substrate.

FIG. 12 displays schematic illustrations demonstrating the easy customization and full reusability of a substrate material of the present invention with different shapes and complex patterns.

FIG. 13 is an image showing a mechanical vibration table.

FIG. 14 is a graph showing the relative resistance variation of a substrate material of the present invention with different thicknesses under low-frequency mechanical vibration interference.

FIG. 15 schematically demonstrates a substrate material of the present invention for circuit repair.

FIG. 16 schematically demonstrates a substate material of the present invention for interaction with a smart phone.

FIG. 17 illustrates an exemplary set up for using a substate material of the present invention and a digit multimeter for detecting human body motion signals.

FIG. 18 is a graph and schematic diagram illustrating the detection of variation in the cyclic motion signals during the bending of a human finger.

FIG. 19 is a graph showing the seal healing property of the substrate material of the present invention.

### DETAILED DESCRIPTION OF PREFERRED EMBODIMENTS OF THE INVENTION

Conventional epidermal electronics have faced both technical and commercial challenges to deliver technically reliable and commercially affordable functionalities. One of the challenges is concerned with the ineffective engagement of such electronics to the skin surface of a human subject.

Epidermal electronics, a type of flexible electronic system, can be applied to the body surface to perform tasks such as monitoring physiological signals, facilitating human-machine interaction, and providing assistive therapy. This type of flexible electronic system can be classified into various forms, including electronic patches, electronic tattoos, and electronic ink, based on their physical characteristics. Despite recent advancements in creating thin and well-integrated electronic patches, they still face limitations regarding their inability to be customized in terms of shape and size for specific applications. Conversely, electronic tattoos offer a solution to this issue by pre-fabricating and tailoring the desired morphology prior to application on the skin. However, the complexity of the fabrication process and the requirement for transfer printing during deployment pose challenges to their conformability with the skin. On the other hand, electronic ink allows for on-demand customization by directly writing onto the targeted epidermal area. Nevertheless, electronic ink typically has a limited shelf life, and exposure to sweat on the body surface can cause its performance deterioration, detachment, or even failure.

In recent years, “Silly Putty-like” material, as a flexible and deformable material, has emerged and been utilized in the applications of soft robotics, circuit reparation, and flexible electronics. This material exhibits enough self-healing and malleability due to its exceptional viscoelasticity, allowing it to be easily shaped and molded to meet specific requirements. “Silly Putty-like” hydrogel, due to its biocompatibility, has sparked a growing interest in its application within the field of wearable electronics. Capitalizing on inherent deformability and bio-adhesive properties, this hydrogel can be flexibly applied to the skin surface without the requirement for printing equipment or auxiliary fixation methods. However, the fabrication process of available hydrogel-based “Silly Putty-like” material with good conductivity is rather complicated and involves the use of high-cost ingredients, and the conductive composites used are still confined to a few options, such as silver/gold nanowires/particles, conductive polymers, MXene, and liquid metals. These existing composites come with certain limitations, including prohibitive cost, limited shape customization, and difficult reusability, hindering the widespread utilization of such materials in the field of epidermal electronics. In addition, although the electrical performance of “Silly Putty-like” hydrogels can be improved by increasing the doping ratio of the conductive active materials to the substrate, their mechanical properties may be adversely affected. Hence, the development of “Silly Putty-like” hydrogels that combine appropriate electrical performance, mechanical performance, adhesive properties, and self-healing capability remains a challenging task. Consequently, “Silly Putty-like” hydrogel, with the ability to overcome these obstacles, holds great promise in achieving electromechanical accessibility, cost-effectiveness, easy customization, and long-term stability for epidermal electronics across a broad spectrum of practical applications.

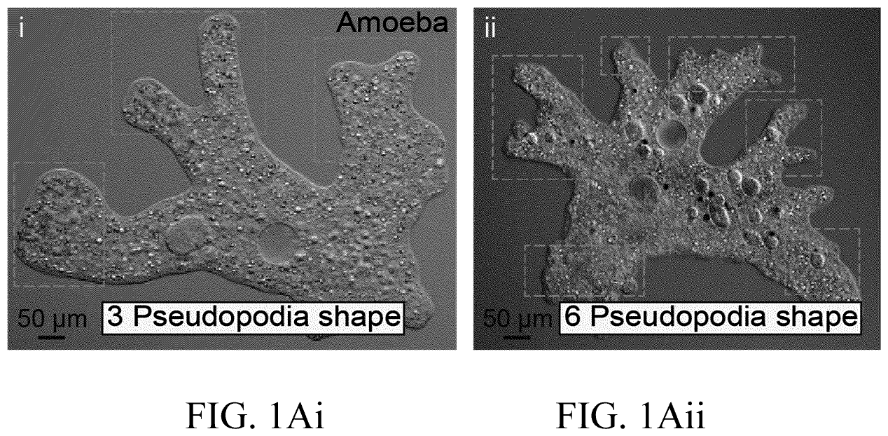

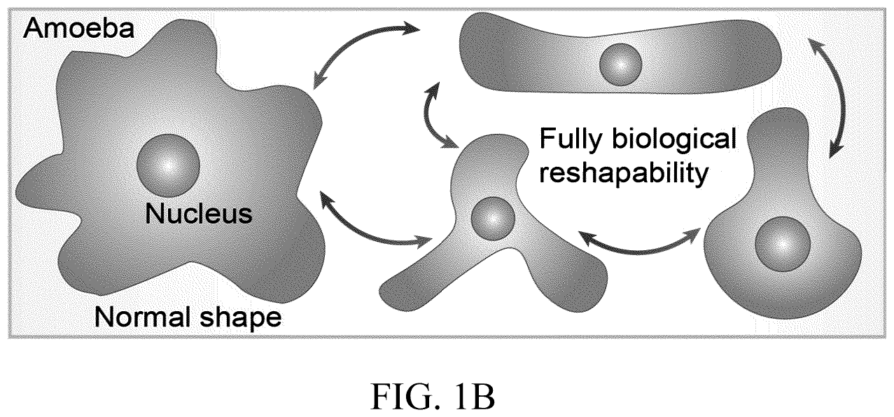

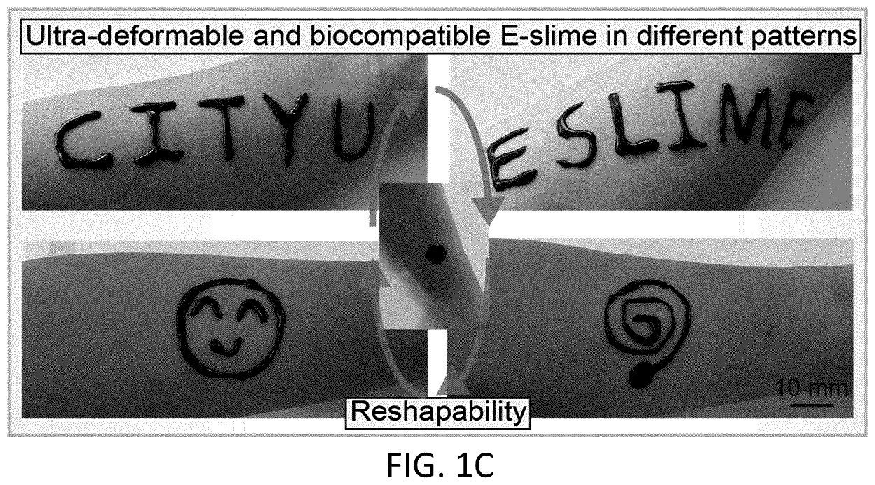

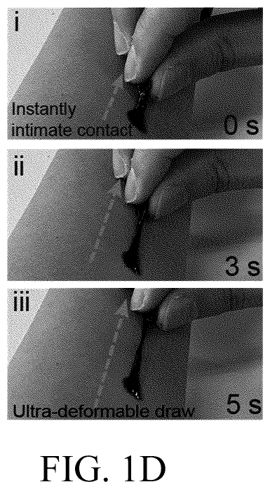

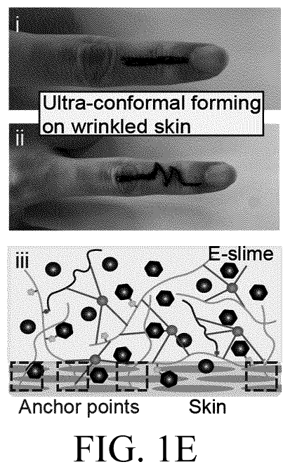

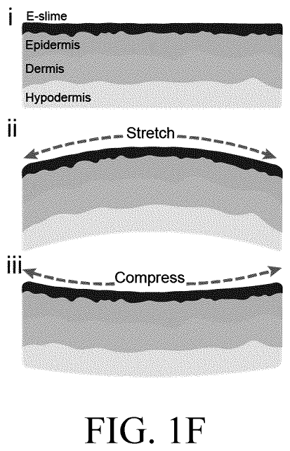

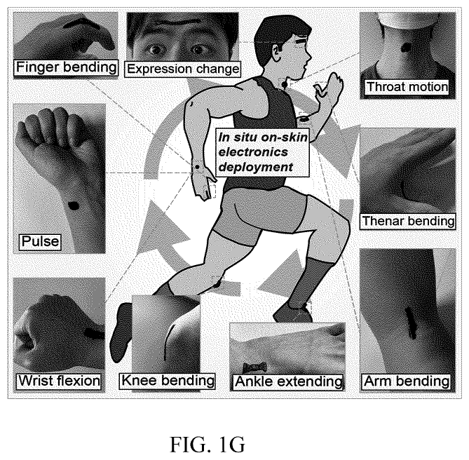

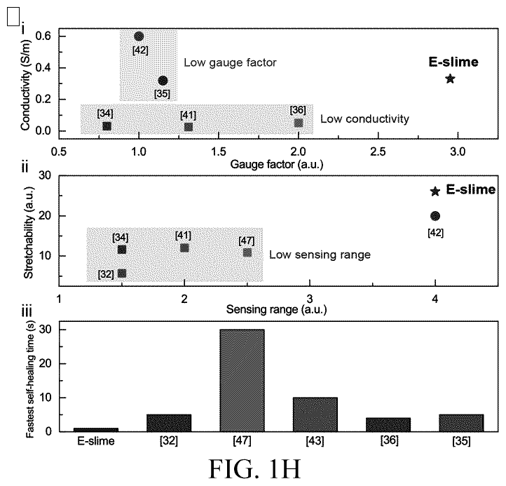

Amoebas, as unicellular organisms capable of promptly altering their shape in response to external stimuli, exhibit remarkable adaptability to their surrounding environment. They move towards a cue on surfaces, accompanied by their change in shape because amoebas extend their cytoplasm and elastic plasmalemma outward through actin polymerization, which leads to the formation of temporary arm-like pseudopods that helps them to stick to the substratum for gaining traction and thus propel themselves forward (see FIG. 1Ai to FIG. 1B).

Studies leading to the present invention led the inventors to study the biological reshapability and environmental adaptability of amoeba which had inspired the inventors to arrive at the present invention. For example, one aspect of the present invention is concerned with a substrate material, sometimes referred to as hydrogel material (or E-slime, a name given for this aspect of the invention), for epidermal use on the subject. Broadly, the substrate material has an ultra-deformable (˜2600% strain), bio-adhesive (adhesive strength ˜3 kPa), strong self-healing (fastest recovery time ˜1s, maximum wound distance ˜5 mm), and electromechanical-durable wearable electronic slime (E-slime) is proposed, which can instantaneously form on-skin electronics in situ to detect body motion and physiological signals. Embodiments of the E-slime demonstrate desired sensing performance with high sensitivity (gauge factor 2.95), wide sensing range (up to 400% strain), and low detection limit (˜1% strain), which can seamlessly adhere to the skin and can be easily reused multiple times (˜100 cycles usage). The E-slime also enables on-the-fly deployment of motion monitoring tasks at various body locations, demonstrating its versatility and reliability for body motion recognition and personal health monitoring. The E-slime is applicable to next-generation green electronics, motion sensing devices, and wearable human-machine interfaces, helping to ensure healthy lives and promote well-being.

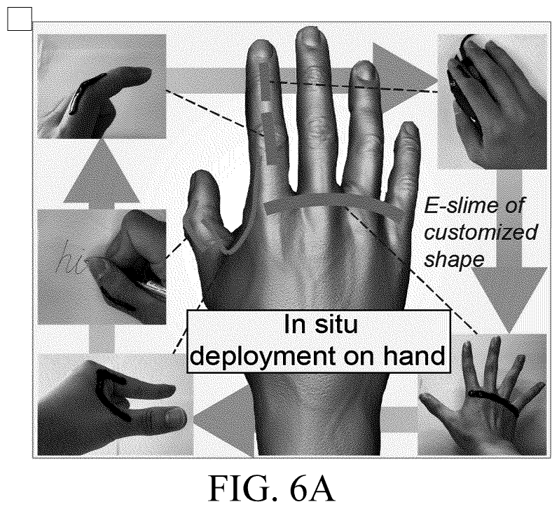

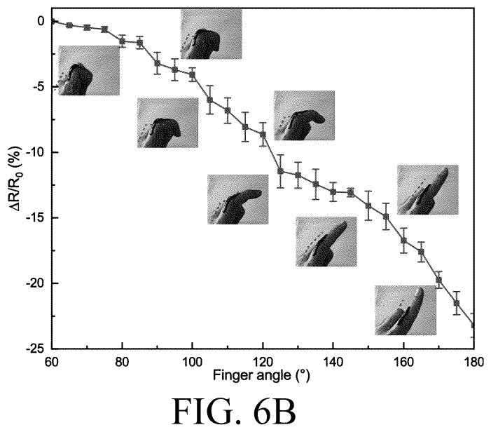

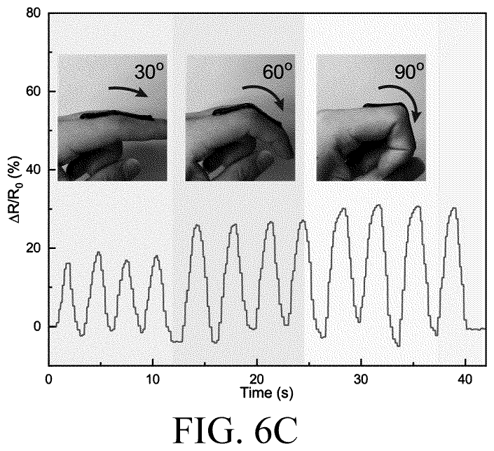

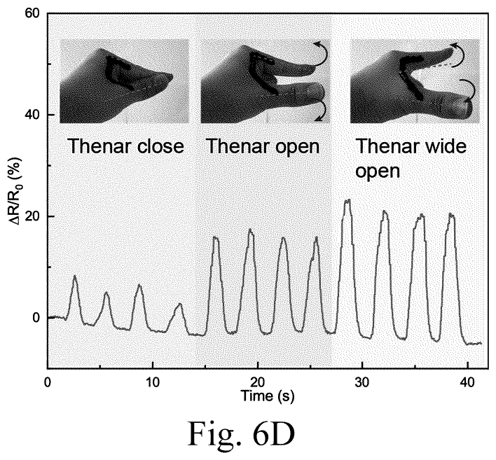

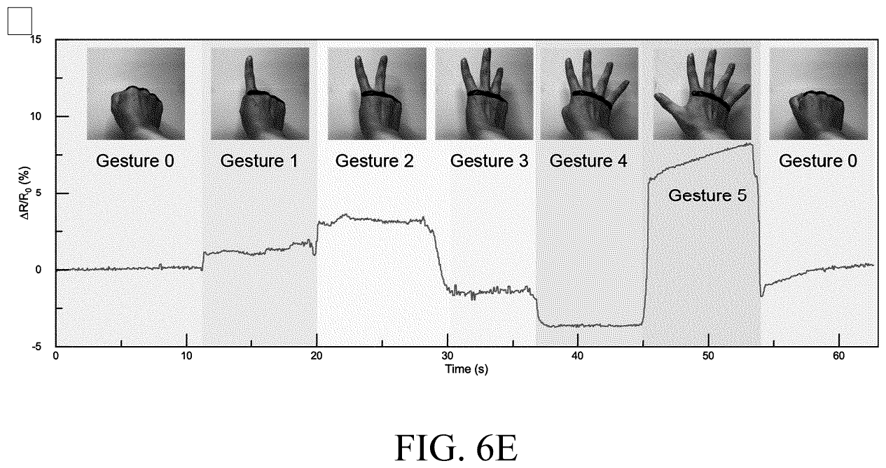

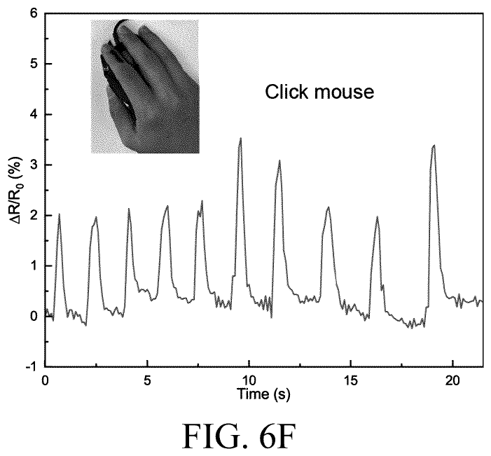

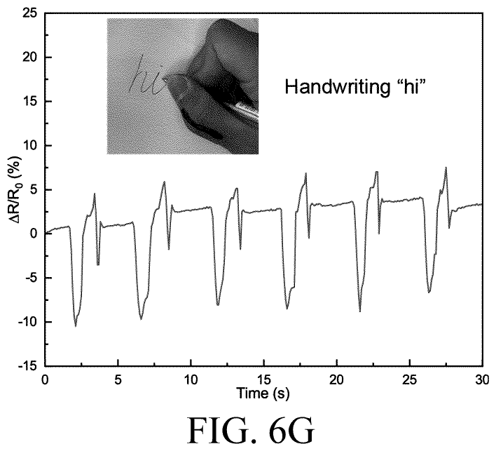

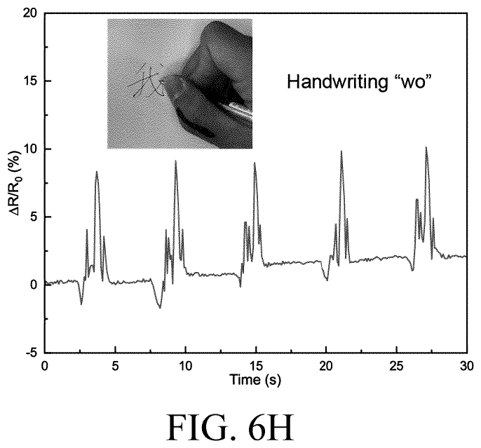

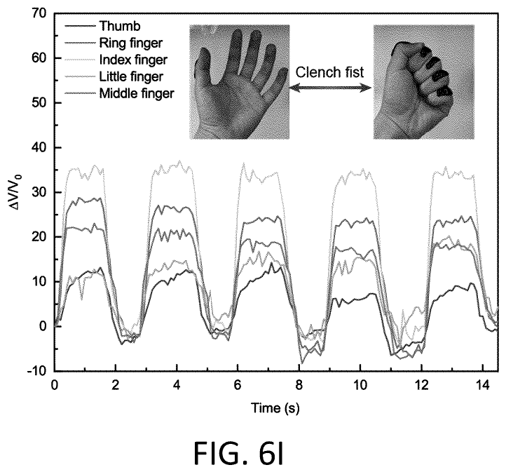

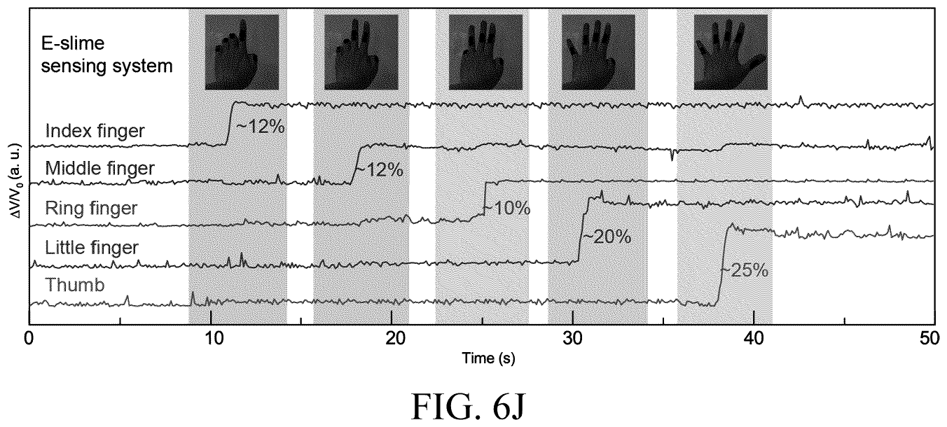

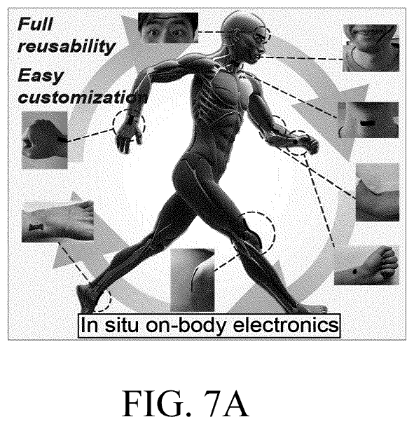

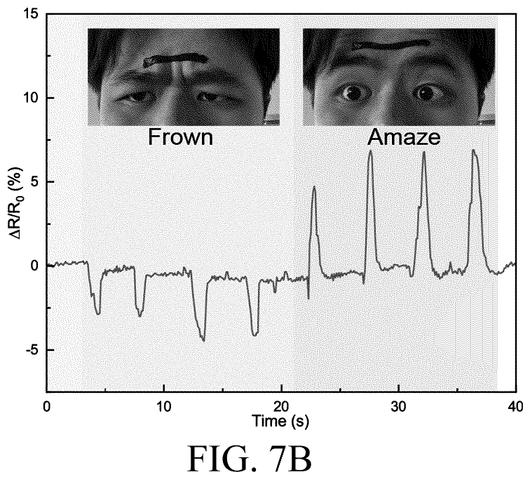

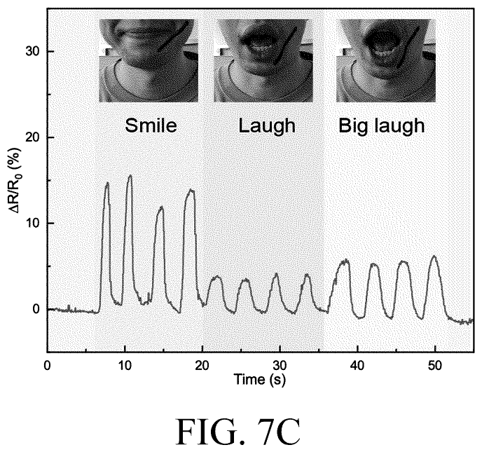

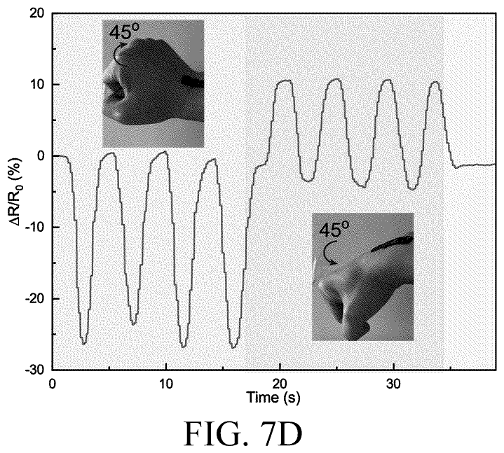

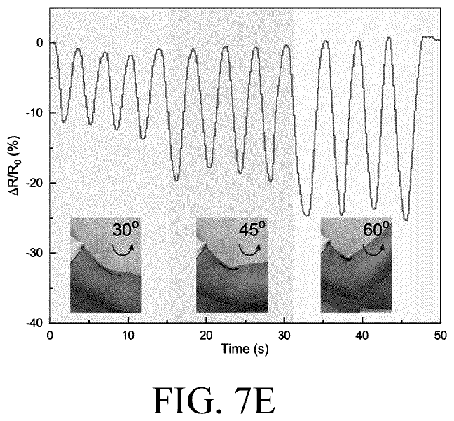

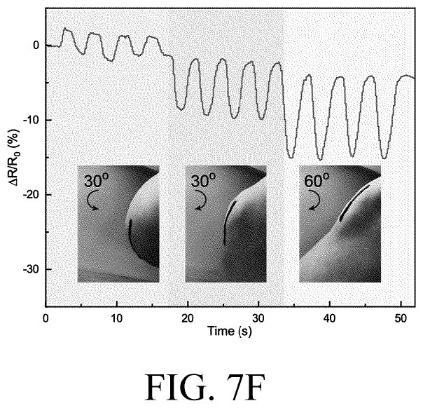

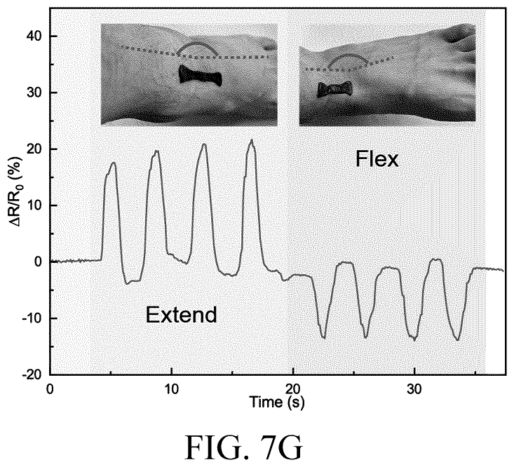

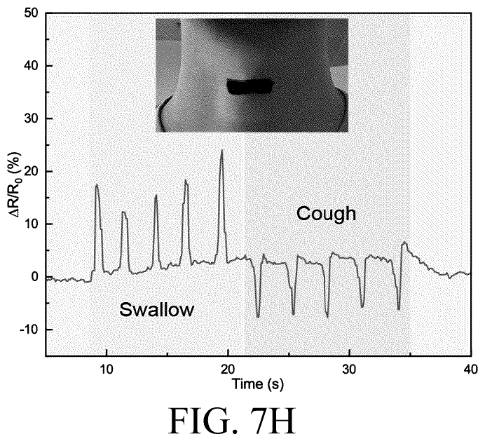

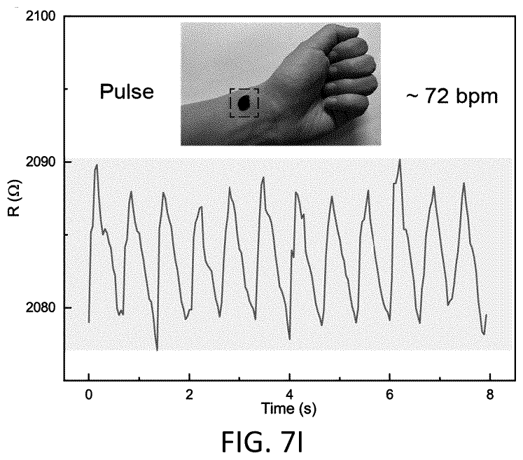

More specifically, aspects of the present invention utilize biocompatible polyvinyl alcohol (PVA) as a matrix and introduce flexible epidermal substrate material (E-slime) through a fabrication method involving tannic acid (TA), glycerol (gly), and cost-effective carbon-based conductive materials (graphite and carbon black). As shown in FIG. 1C, the substrate material, as a type of hydrogel, can undergo deformation in multiple different patterns on the skin due to its fully reusable and easily customized properties. Benefiting from the ultra-conformal ability, the substrate material can instantly form electronic skin in situ on the targeted epidermal area tightly, even on the wrinkled skin (see FIG. 1D) due to the rigid anchor points constructed by inner crosslinking of the substrate material (FIG. 1E). With the engagement of the substrate material on the epidermal area, the signals associated with the skin motion can be obtained (FIG. 1F). Furthermore, due to its moderate elastic modulus (˜5.3 kPa) and solid strength (up to ˜2 kPa for ˜40 cycles), the substrate material can be easily peeled off from the attached area, reshaped into a different form, and re-deployed for sensing in a new target area. The substrate material exhibits the ability to withstand external mechanical disturbances due to its robust self-healing cycle (see FIGS. 6-7) and repeatable self-healing capability, expanding its range of applications under dynamic conditions. As illustrated in FIG. 1G., the substrate material can be conveniently deployed at various locations on the body surface to measure various human motion signals, including joint extension and flexion, throat movements, facial expressions, and pulse. These demonstrations further validate the multifunctionality, reliability, and applicability of the substrate material in personal health monitoring and body motion recognition. Moreover, FIG. 1H indicates that the substrate material can exhibit desired conductivity (0.33 S/m), gauge factor (2.95), sensing range (up to 400%), stretchability (˜2600%), and self-healing time (˜1 s) in comparison to similar/conventional “Silly Putty-like” hydrogels offering comparable information. Additionally, more performance comparisons (detection limit ˜1% strain, maximum self-healing distance up to ˜5 mm, and self-healing cycles ˜30 times) provided in Table 1 highlight the substrate material's holistic blend of appropriate electromechanical properties, reusability, and resilient self-healing capacity. The present invention allows the application of next-generation artificial electronic skin, green electronics, intelligent medical sensing devices, and wearable human-machine interfaces. Further details, including information on experiments leading to the present invention conducted, are depicted below.

### Results and Discussion

### Schematic Fabrication, Crosslinking Structure, Conductive Mechanism, and Structural Characterizations of Substrate Material

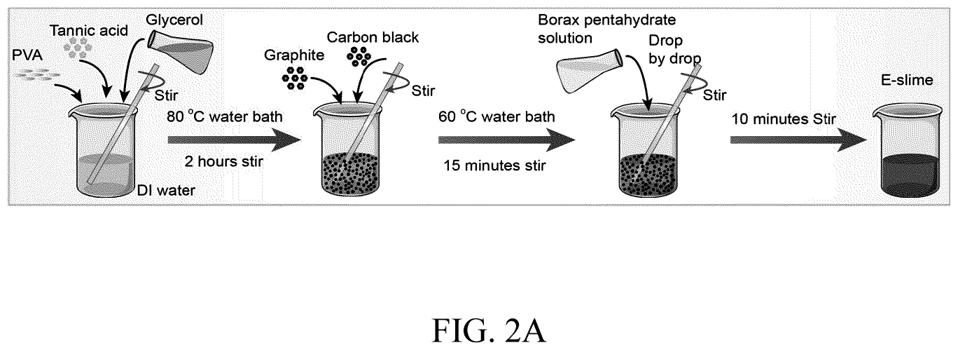

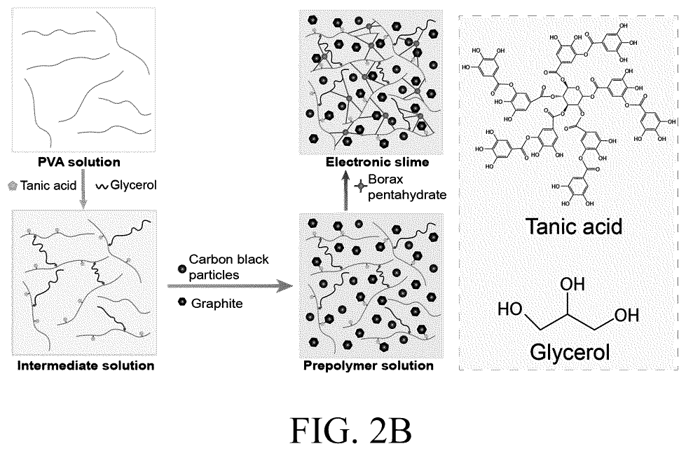

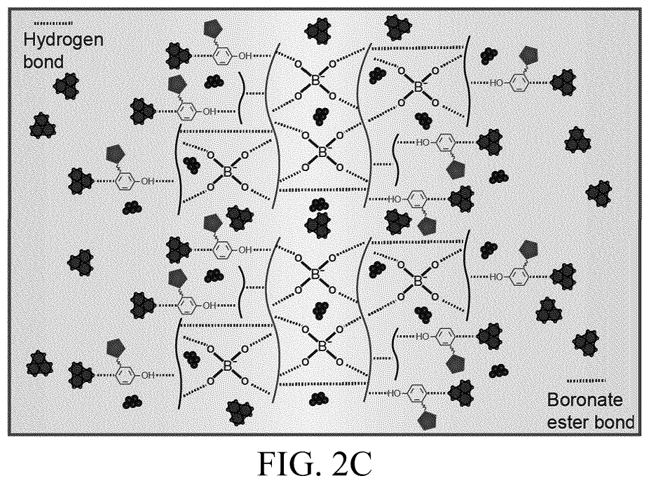

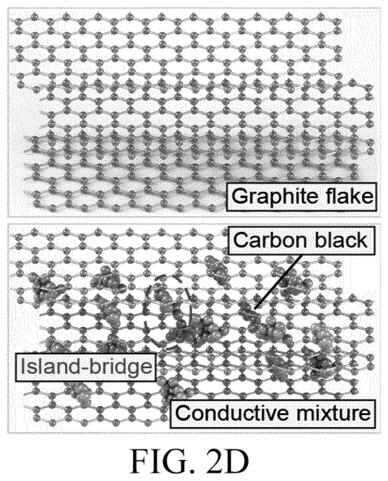

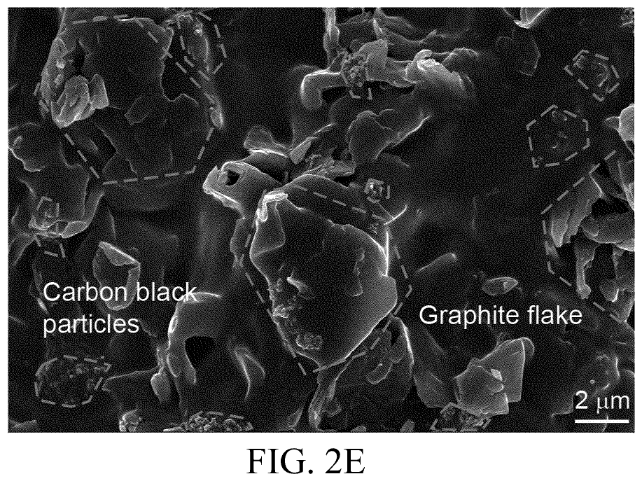

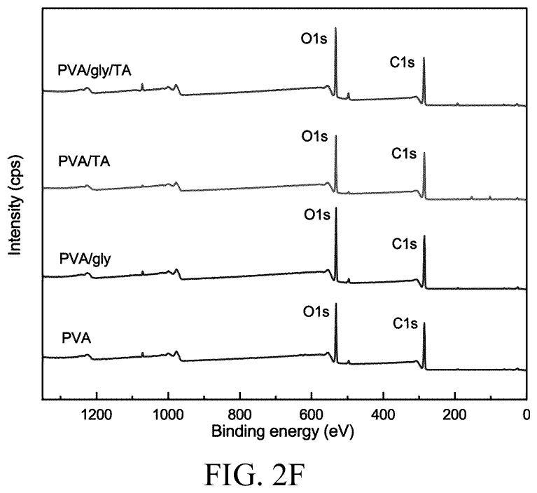

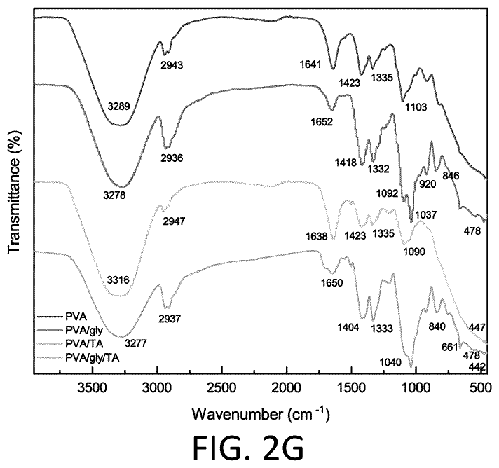

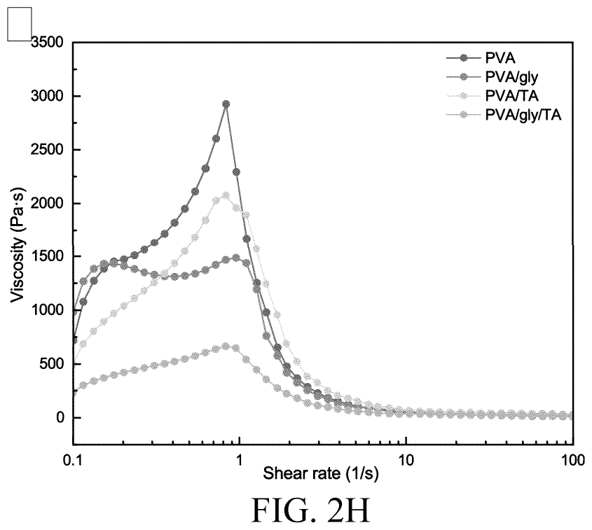

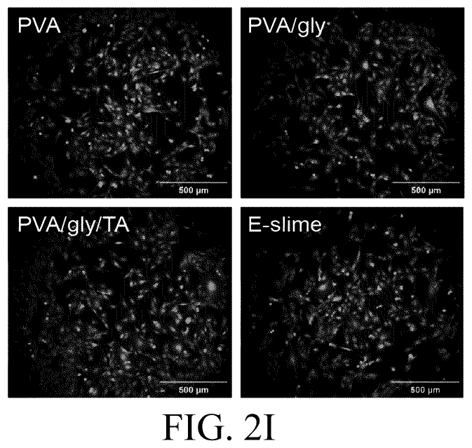

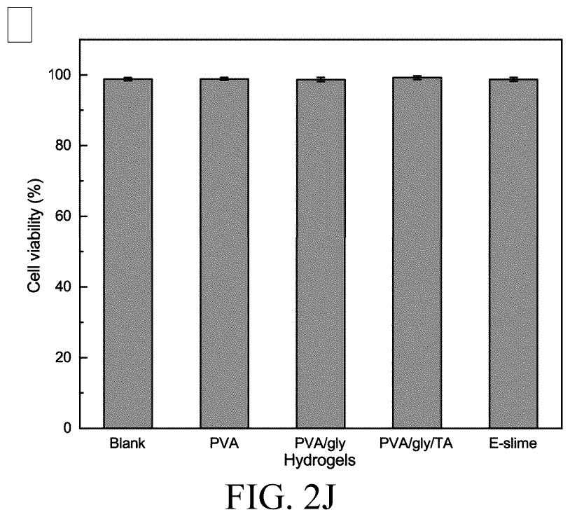

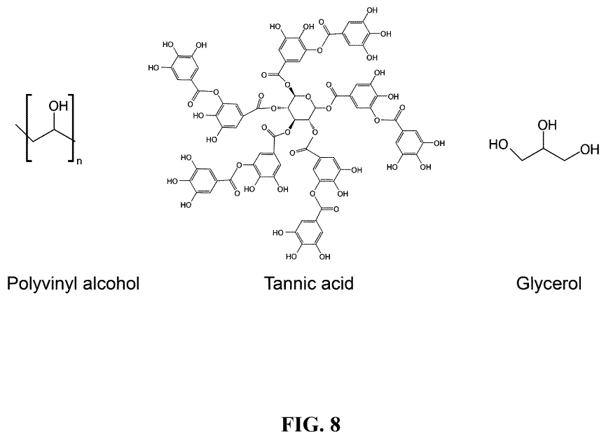

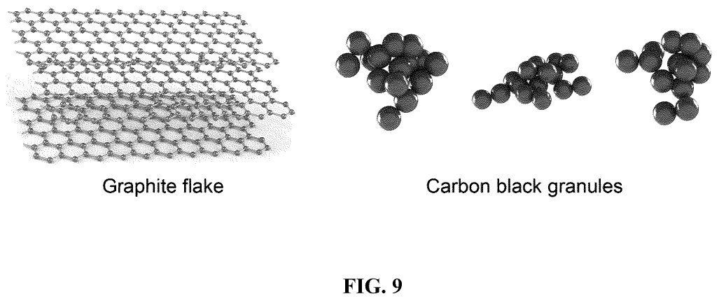

PVA, a synthetic polymer with biocompatibility, is employed. PVA is accompanied by easy-to-obtain, low-cost, and environmentally-friendly raw composites shown in Tables 2-3. IPVA is used as the matrix material in the preparation of the substrate material E-slime in three steps or in three main steps, as depicted in FIG. 2A. In some embodiments, a mixed solution is firstly prepared with PVA, glycerol, and TA (see FIG. 8) in a predetermined concentration in an 80° C. water bath for 2 hours stirring. After the dissolution of all chemicals, a conductive material of graphite and carbon black powder (see FIG. 9) is added to the mixture in a 60° C. water bath. Although graphite and carbon black are not soluble in water, TA monomer is able to undergo oxidative polymerization to form deposition onto the surface of graphite, while carbon black is dispersed in the interlayer spacing due to the high reduction ability of modified graphite by TA, achieving uniform mixing. Then, a borax pentahydrate solution is added drop by drop into the conductive mixture with continuous stirring to generate a substrate material of the present invention.

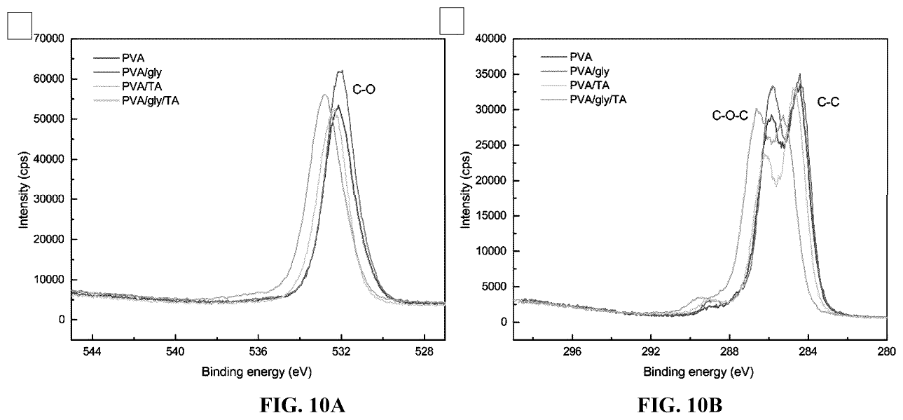

FIG. 2B illustrates the molecular crosslinking process in the synthesis of the substrate material. The PVA solution is dissolved with glycerol and TA, forming the hydrogen bonds between catechol groups, respectively. Graphite and carbon black, as conductive particles, are doped and evenly distributed into the intermediate solution to create a prepolymer solution. To obtain the substrate material, borax pentahydrate plays a role as a chemical crosslinker, inducing the polymerization of the prepolymer mixture. As indicated in FIG. 2C, the establishment of a hierarchical three-dimensional hydrogel network in the substrate material is facilitated by the intertwining of polymer chains across various substances, the presence of dynamic borate ester bonds between different components, and the concurrent occurrence of intramolecular and intermolecular hydrogen bonds. To improve the conductivity of the material, carbon black granules are incorporated as a filler into the interlayer spacing of the graphite sheet to establish the island-bridge structure (FIG. 2D). Carbon black has an amorphous, isotropic structure, allowing for uniform conductivity in all directions. Graphite, with its layered structure, has high conductivity within each layer but lower conductivity between layers. Consequently, the conductivity can be enhanced by combining graphite and carbon black in a suitable ratio, which helps to bridge the graphite planes with the carbon black particles. The amorphous hydrogel structure of the substrate material was characterized by scanning electron microscopy (SEM), X-ray photoelectron spectroscopy (XPS), Fourier transform infrared spectroscopy (FTIR), and viscosity test. The microstructure of the substrate material imaged by SEM indicates that the hydrogel is relatively compactly crosslinked with the establishment of the island-bridge structure (graphite and carbon black), as shown in FIG. 2E. XPS spectra show that all four hydrogels have similar peaks (FIG. 2F), and the high-resolution XPS of O 1s and C 1s (FIG. 10A and FIG. 10B) indicates the appearance of C—O, C—O—C, and C—C due to these materials' formation (C, H, and O). C—O bonds suggest interactions between PVA and borate, indicating crosslinking, while C—O—C linkages confirm the integrity of PVA's polymeric backbone. C—C bonds indicate the presence of stable carbon frameworks within the organic components of the hydrogels. FTIR spectroscopy illustrates the interaction and structure formation among PVA, glycerol, TA, and borax (FIG. 2G). The peak at 1333 cm⁻¹ was assigned to the C—O stretching mode of the ester part and the —OH bond plane vibration, respectively. These peaks are typical absorption features of TA and indicate its successful combination with other components in the substrate material. The peak at 1040 cm⁻¹ was primarily attributed to C—O stretching vibrations, which can indicate the extent of cross-linking within glycerol and PVA of the hydrogel. The small peak at 1404 cm⁻¹ is caused by the bending of the B—O—B bond in the borate network, confirming the presence of crosslinking between borax and the PVA chain, forming a boronic ester bond.

Additionally, FIG. 2H shows that substate material exhibits a low viscosity by comparing the viscosity curve of different hydrogels based on PVA, indicating E-slime's sticky and thick consistency at low shear rates. When the shear rate increases from 1 s⁻¹, the substrate material illustrates a typical shear-thinning behavior owing to a rapid decrease of the viscosity from 665 Pa·s, verifying its property as a non-Newtonian hydrogel. The cell viability test of these hydrogels was conducted as shown in FIG. 2i (experimental setup in Experimental Section). FIG. 2J shows that the cell viability of E-slime is 98.72%, validating its biocompatibility. These characteristics demonstrate that the substrate material is a highly flexible hydrogel, possessing a certain plasticity, showing the potential to be a desired candidate for epidermal electronics.

### Mechanical and Electrical Characterizations of the Substrate Material

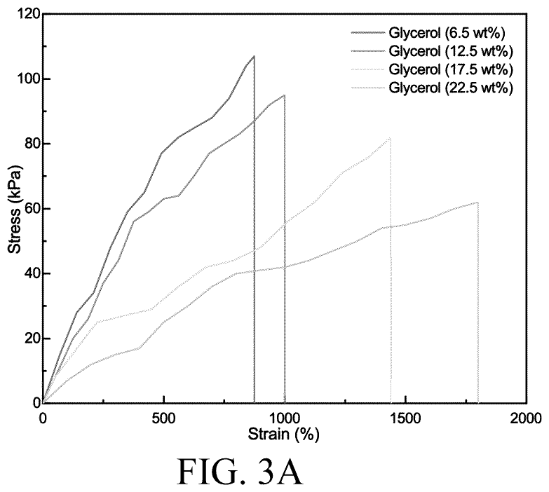

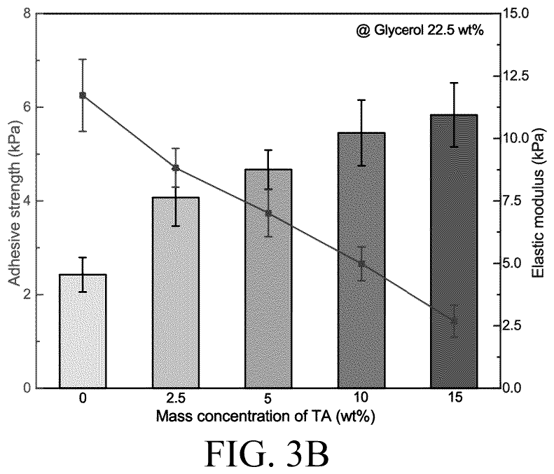

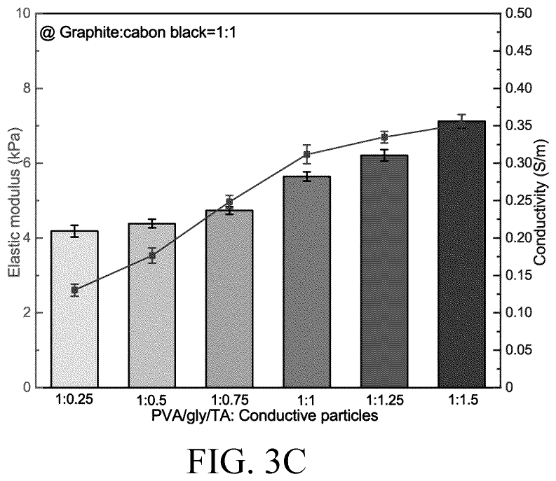

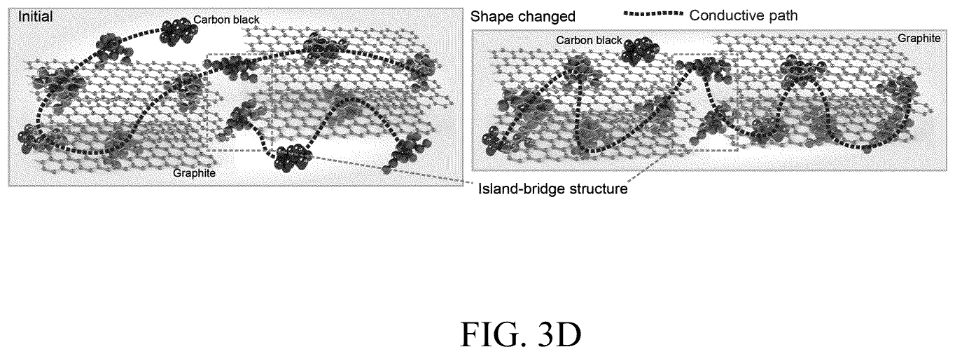

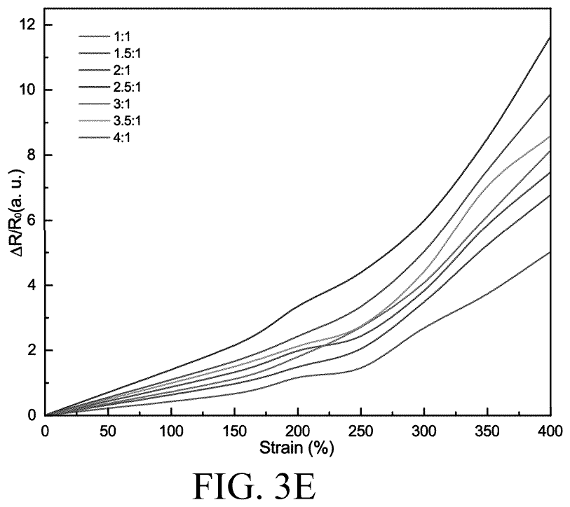

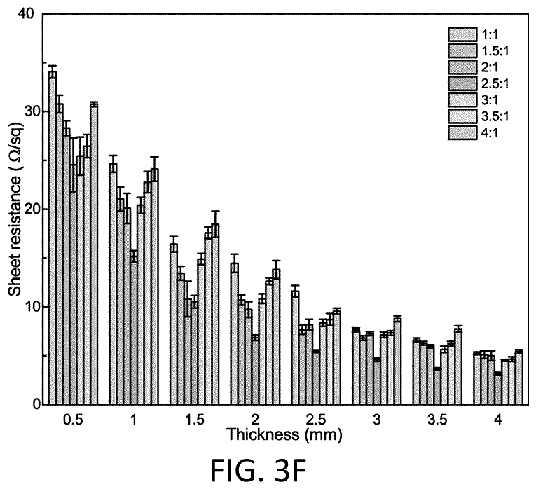

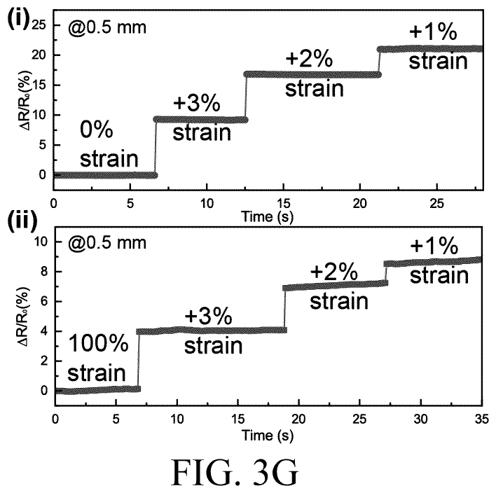

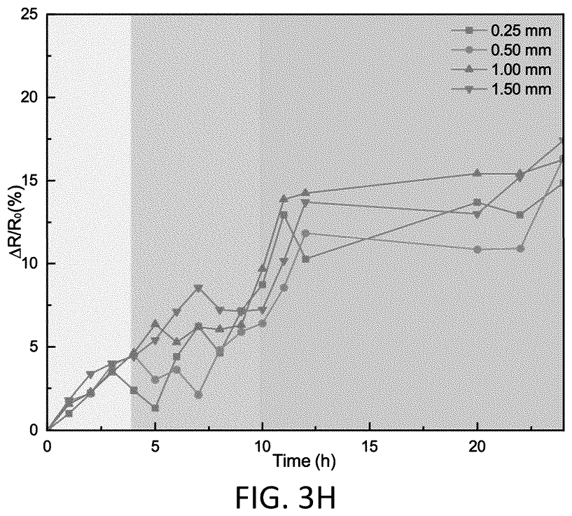

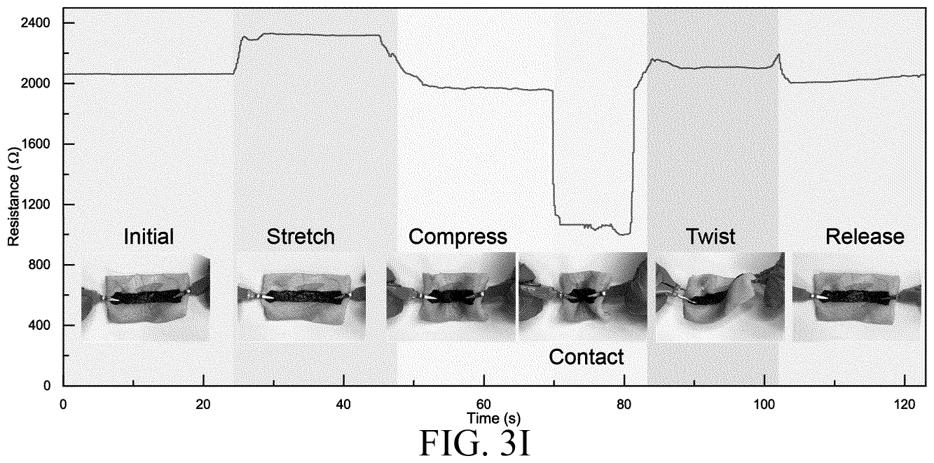

To investigate the glycerol's influence on the mechanical performance of PVA hydrogel, PVA hydrogel containing glycerol with different quality (6.5 wt %, 12.5 wt %, 17.5 wt %, and 22.5 wt %) were made for tensile experiments. As illustrated in FIG. 3A, with the increase of glycerol content, the tensile strength of the substrate material increases significantly from ˜60 kPa to ˜110 kPa, while elongation at break decreases from ˜1800% to ˜875%. In addition, the tensile strength and elongation at break have consistent changes when glycerol content increases from 17.5 wt % to 22.5 wt %. The results suggest that as the amount of glycerol increases, the crosslinking density between different components also increases, resulting in the fracture of numerous hydrogen bonds to dissipate energy. Consequently, this leads to a decrease in the elongation at the breaking point of the hydrogel. Thus, PVA/gly hydrogel possesses the desired stretchability and mechanical strength with a mass concentration of 22.5 wt %. TA is a plant-derived polyphenolic substance that contains abundant catechol groups. As shown in FIG. 3B, the experiments were conducted to evaluate the mechanical effect of TA on PVA/gly hydrogel. With the increased content of TA from 0 wt % to 15 wt %, the adhesive strength of PVA/gly/TA hydrogel increases from 2.42 kPa to 5.84 kPa, while its elastic modulus decreases from 11.72 kPa to 2.67 kPa. The results indicate that TA makes PVA/gly hydrogel more flexible and improves its adhesive ability. It is noteworthy that when the mass concentration of TA exceeds 15 wt %, the crosslinking process becomes unachievable, potentially due to the occurrence of aggregation between TA and PVA caused by an excessive amount of catechol moieties in TA. Thus, the mass concentration of TA is fixed at 10 wt % for subsequent research, where the hydrogel demonstrates robust adhesive strength (7.07 kPa) and possesses a suitable elastic modulus (5.45 kPa). To investigate the formal ratio of conductive particles (graphite and carbon black) to PVA/gly/TA hydrogel, the elastic modulus and conductivity of the prepared testing samples were evaluated, as illustrated in FIG. 3C. With the increase of conductive dopant content, the elastic modulus and conductivity of the substrate material increase, respectively. At the mass ratio of 1:1 (PVA/gly/TA hydrogel:conductive particles), the substrate material has the appropriate elastic modulus (5.65 kPa), and the conductivity (0.31 S/m) has a sharp increase from a mass ratio of 1:0.25 to 1:1 while a slow upward trend when the mass ratio increases from 1:1 to 1:1.5. Based on the above analysis, the mass ratio of 1:1 is chosen to fabricate the substrate material for following studies.

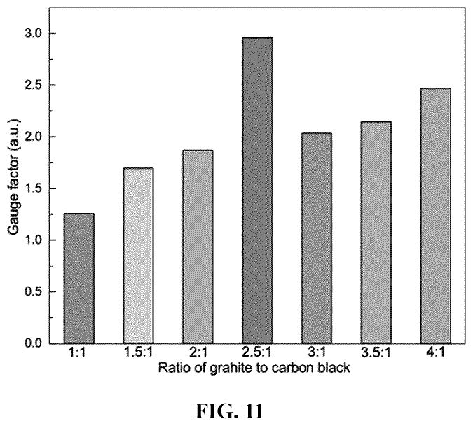

FIG. 3D illustrates the sensing mechanism of the substrate material. Carbon black particles act as a filler to create an island-bridge structure through conductive paths, enhancing the composites' overall conductivity by bridging the gaps between graphite layers and different graphite. When the substrate material changes its shape, the internal structure of carbon-based composites will also be impacted, resulting in enhancement or diminishment of the conductive paths due to the change of composite location, consequently altering the electrical resistance of the substrate material. Furthermore, to evaluate the effect of the carbon material composition ratio on the conductivity of substrate material, the relative resistance variation of the substrate material was characterized by different mass ratios of graphite to carbon black. As shown in FIG. 3E, the relative resistance variation changes positively with the increase of applied strain due to the effect of the elongated internal electron transfer channels. Moreover, with the rise of the mass ratio (graphite:carbon black), the average gauge factor of the corresponding substrate material exhibits a trend of decreasing first and then increasing (FIG. 11). The highest gauge factor (˜2.95) occurs at the mass ratio of 2.5:1; continuing to expand the graphite content will reduce the connectivity of the carbon black, hindering the sensitivity, which is consistent with the aforementioned theoretical analysis. In addition, the sheet resistance with different thicknesses of E-slime (different mass ratios of graphite:carbon black) is further investigated (FIG. 3F). Similarly, the sheet resistance presents a decreasing and then increasing trend with the mass ratio's increase no matter what thickness E-slime has, which is in accordance with the results of the gauge factor. Therefore, the ratio of graphite to carbon black is selected as 2.5:1 as the optimal ratio for the substrate material in the subsequent study.

FIG. 3G shows the strain resolution of the substrate material under different strain loads (0 and 100% strain), illustrating that each strain increment correspondingly triggers a noticeable resistance change even with different reference strains. The electrical stability is a crucial parameter for the sensor made by hydrogel, which was investigated by evaluating the resistance change over time (room temperature), as shown in FIG. 3H. All test samples (four different thicknesses) exhibit a resistance variation of less than 5% within the first 4 hours, gradually increasing to around 10% until the 10th hour. This result indicates that the sensor possesses essential electrical performance stability, making it suitable for relatively long-term storage and use. To further examine the durability of the electrical properties of the substrate material under mechanical stress, an analysis of the resistance of the substrate material applied onto porcine skin (formation of in-situ epidermal electronics) was conducted. The pigskin was subjected to various forms of deformation, including stretching (˜20%), compression (˜60°), contact, and twisting (˜50°), to assess its stability. As depicted in FIG. 3I, apart from the resistance increase (˜19%) observed during the stretching process and the resistance decrease (˜50%) during the contact process, the resistance remained consistently around 2000Ω. These findings indicate that the substrate material maintains strong adhesion and stable electrical conductivity, even when applied to an irregular and wrinkled skin surface and in the presence of different types of deformation.

### Easy Customization and Full Reusability of Substate Material

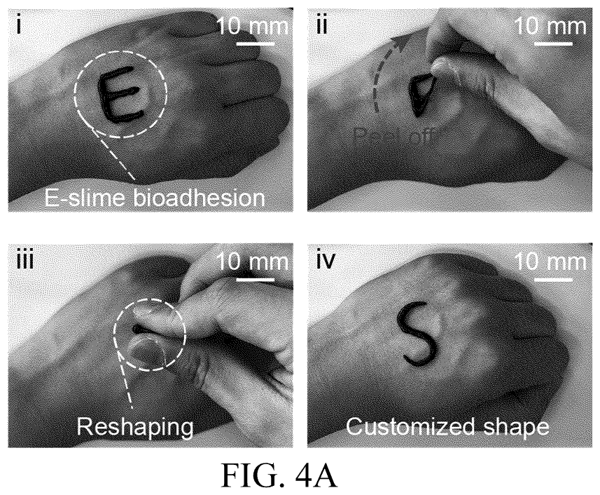

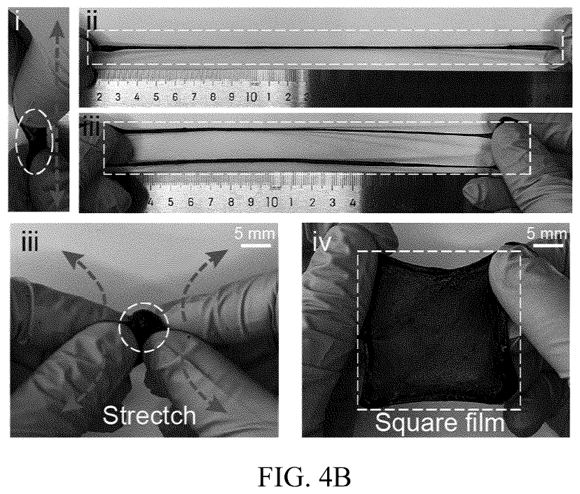

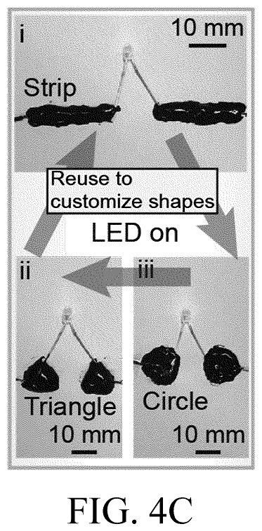

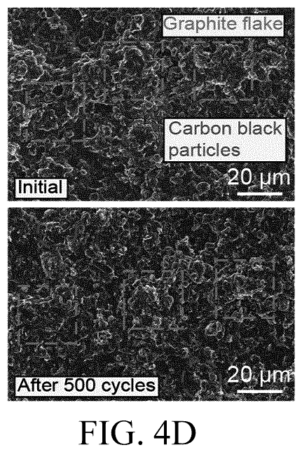

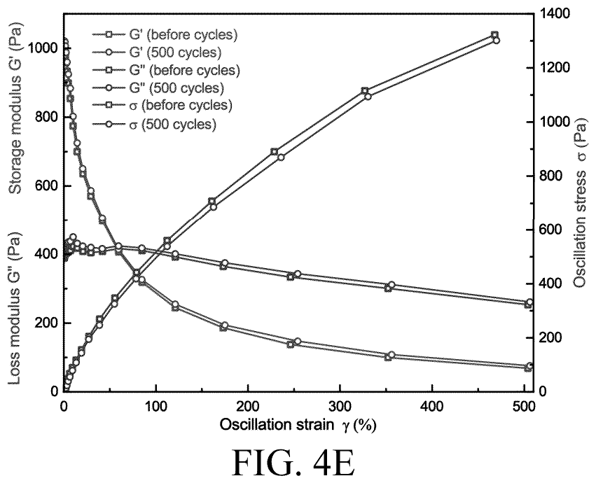

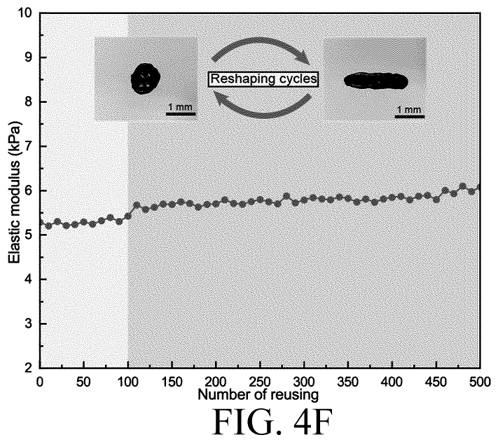

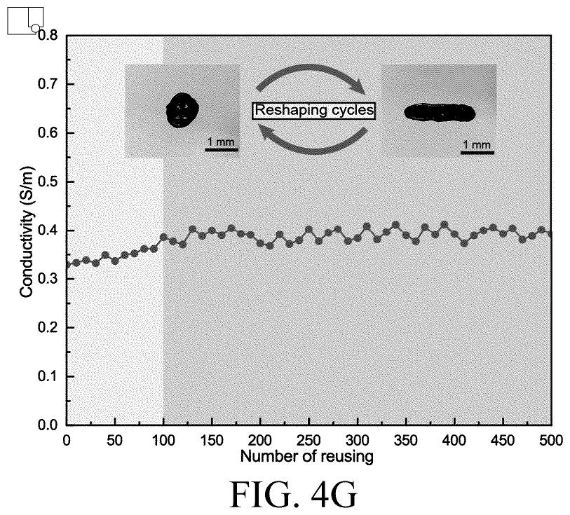

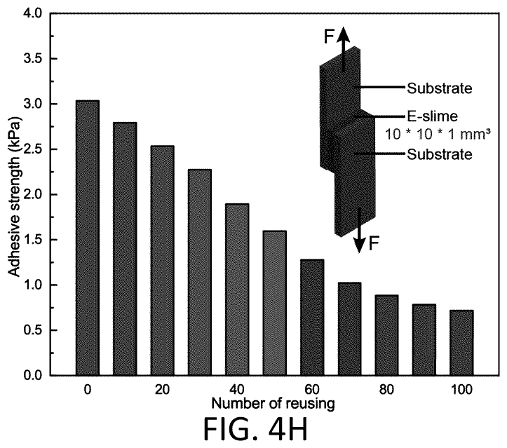

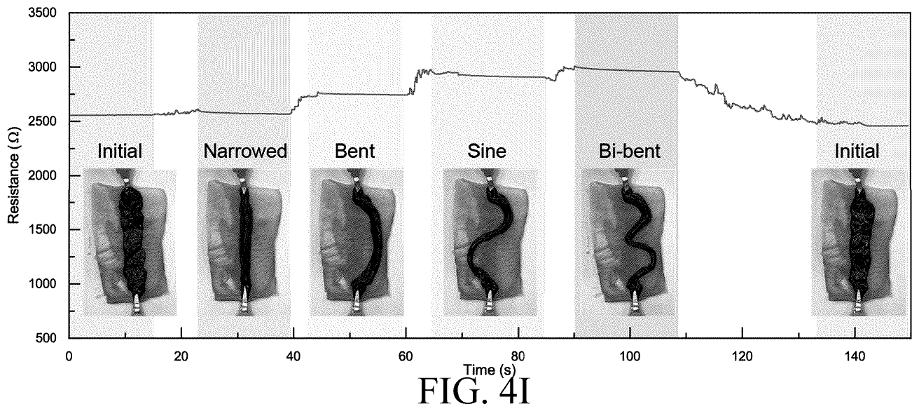

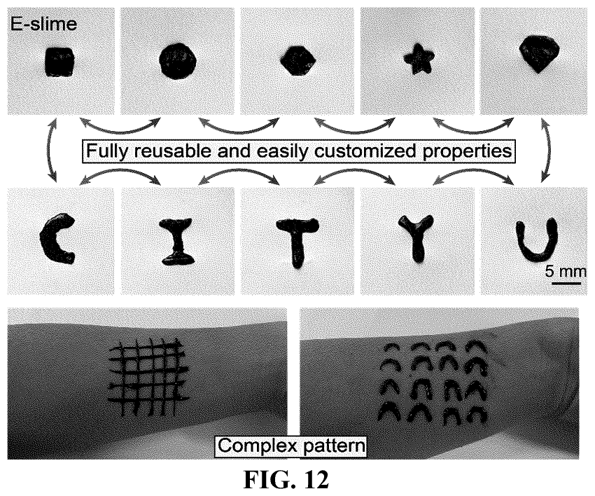

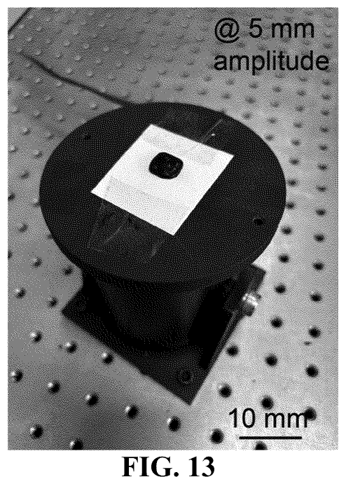

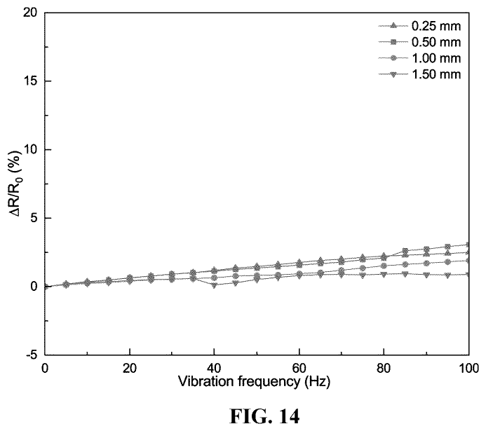

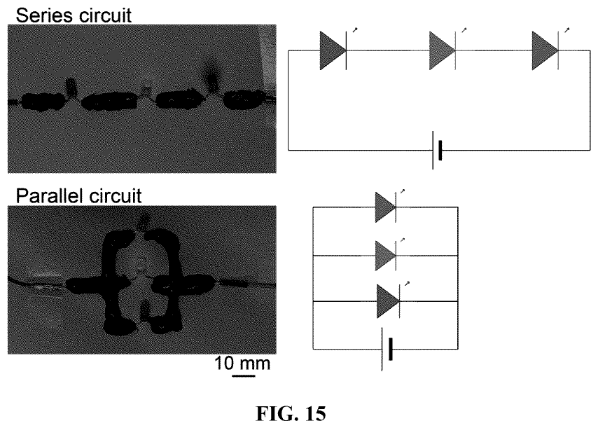

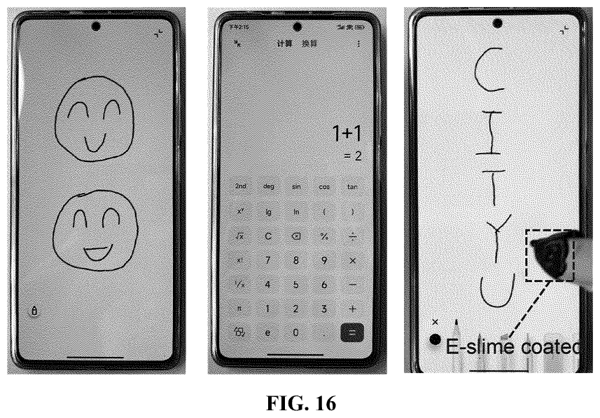

FIG. 4A illustrates the procedure of the substrate material's deployment and utilization for on-skin electronics. The substrate material is easy to apply and adhere to the skin due to its bio-adhesive ability, while it is also effortless to peel off, reshape, and reapply onto the skin in a demanded shape (more shapes and patterns in FIG. 12). Additionally, the impressive tensile strength of E-slime was demonstrated in the stretching test (up to ˜2600% stretching without fracturing), as depicted in FIG. 4B. Its notable mechanical properties also enable E-slime to form a thin film (thickness ˜400 μm), making it advantageous for its utilization as a strain sensor. As presented in FIG. 4C, a piece of the substrate material is connected to illuminate LED bulbs successively in different shapes (strip, triangle, and circle), demonstrating its electrical conductivity with full reusability and easy customization. The result validates that the substrate material is a suitable conductive material for emergency circuit repairs and flexible electronic devices (FIG. 15, FIG. 16). To confirm that the substrate material can maintain its microstructure, the SEM images (initial state without use and 500 cycle uses) were obtained, as shown in FIG. 4D. The graphite flakes and carbon black granules remain dispersed homogeneously in the crosslinked networks after repeated use 500 times, indicating E-slime's reusable stability. Furthermore, the rheological tests were conducted by evaluating the loss modulus, storage modulus, and oscillation stress before and after 500 times use (FIG. 4E). At 250% oscillation strain, the loss modulus (˜333 Pa) is more extensive than the corresponding storage modulus (˜137 Pa), whether before or after use. Moreover, before and after use, the curves of storage modulus and loss modulus remain almost unchanged, respectively. The results suggest that the substrate material is elastic and soft even after continuous cycled usage, which guarantees its easily customized capability.

Furthermore, tests of the substrate material E-slime's reusable electromechanical performance stability were conducted. FIG. 4F presents the elastic modulus of the substrate material under different usage cycles, showing that the elastic modulus gradually increases from 5.29 kPa to 6.09 kPa after repeated use while remaining at approximately 5.3 kPa under 100 cycles. The results suggest that the mechanical performance of the substrate material will remain stable (variation less than 2.5%) with less than 100 uses. Additionally, the electrical stability of E-slime is evaluated in FIG. 4G, exhibiting a slow climbing trend (conductivity increases from 0.33 S/m to 0.39 S/m) less than 100 cycles, then a fluctuation (around 0.26 S/m) after 100 cycles. The findings indicate that the electrical performance of E-slime will also remain stable with less than one hundred uses. In addition to the electromechanical stability for reusable cycles, the reusable adhesion is also assessed, as shown in FIG. 4H. The adhesive strength of E-slime progressively decreases during the using cycles from 3.03 kPa (initial) to 0.72 kPa (at 100 cycles), indicating that although repeated use can reduce the adhesiveness of the substrate material, the adhesive strength remains at a permissible level suitable for skin adhesion.

To further verify the easy customization and full reusability of the substrate material under various postures by in situ formation, an analysis of the resistance of the substrate material applied onto porcine skin was conducted. The substate material was subjected to multiple forms of deformation, including narrowing, bending, sine, and bi-bending, to evaluate its immediate strain response. As illustrated in FIG. 4I, E-slime will obtain the corresponding different resistance response obviously during the whole shape-changing process and will receive the same response as the initial state after reshaping itself back. The aforementioned experimental results collectively demonstrate that the substrate material exhibits suitable mechanical and electrical performance stability for repeated use (up to 100 times) while maintaining adequate bonding strength for repeated adhesion to wrinkled skin. This confirmed repeatability validates the convenient customization and full reusability of the substrate material, further substantiating its viability as an in-situ deployable electronic skin.

### Adhesive and Self-healing Properties of Substate Material

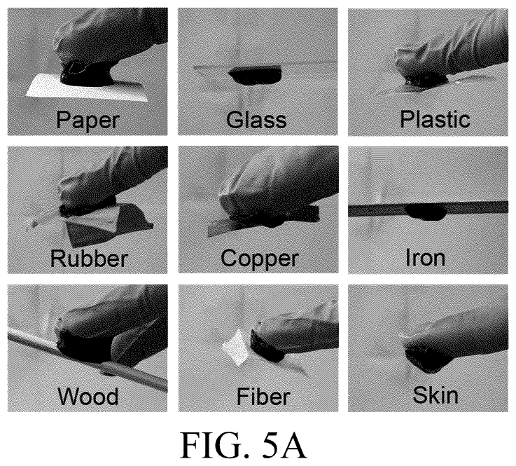

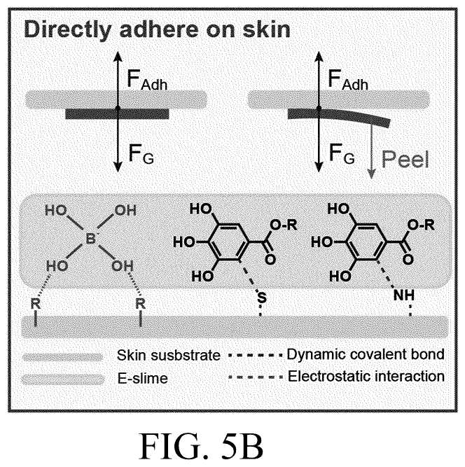

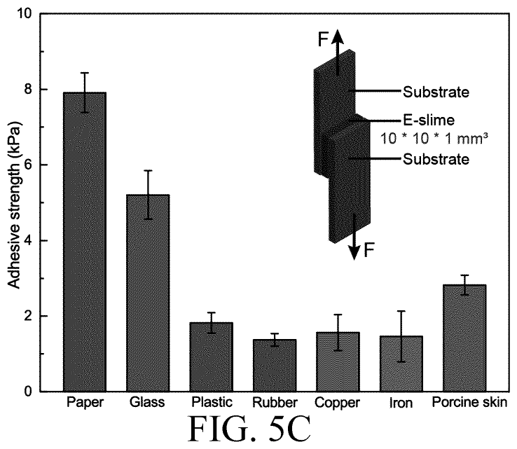

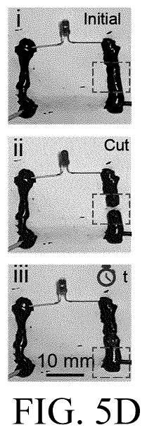

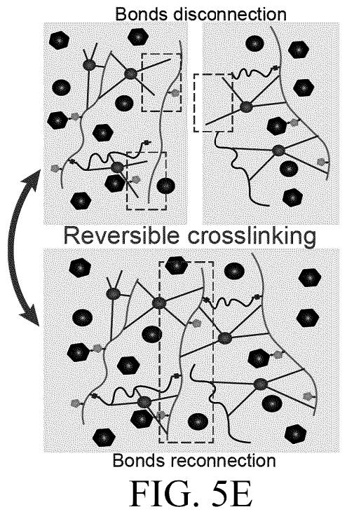

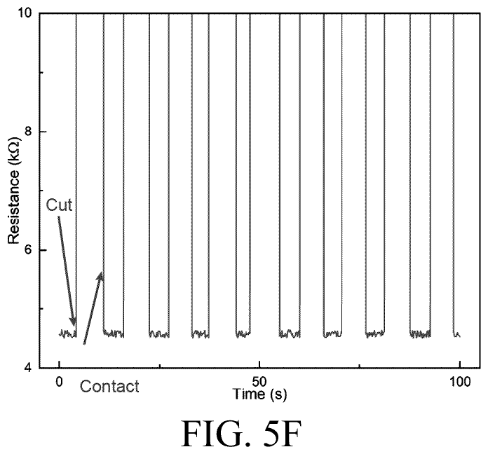

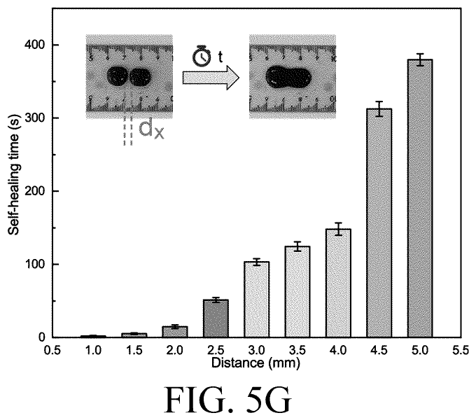

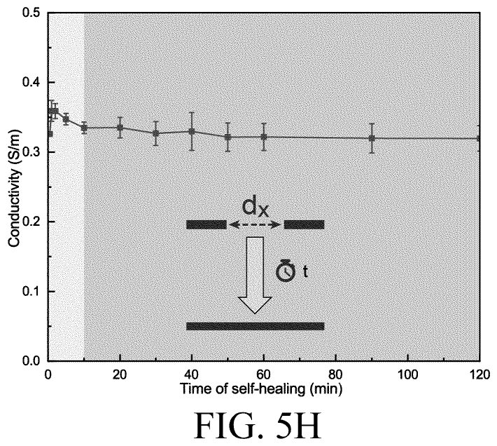

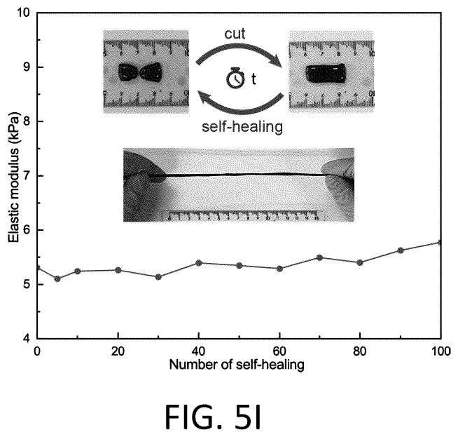

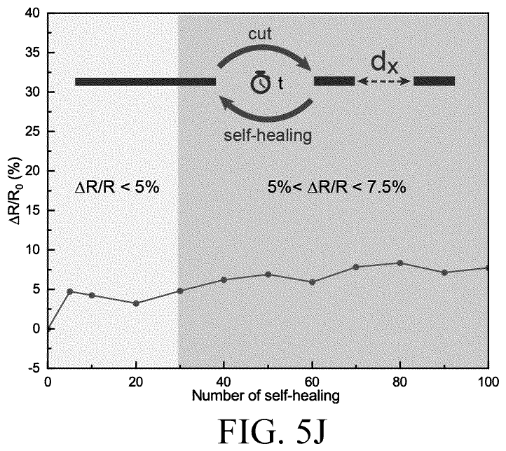

Mussels are able to adhere to rocks and other uneven surfaces due to the presence of specialized proteins (containing catechol groups) in mussel foot secretions. Taking inspiration from the adhesive properties of mussels, the substrate material demonstrates remarkable strength and durability in adhesion. This can be attributed to the abundant presence of catechol groups within the hydrogel matrix from TA, enabling robust and long-lasting adhesiveness on human skin. FIG. 5A depicts that E-slime is able to adhere firmly to various substances, including paper, glass, plastic, rubber, copper, iron, wood, fiber, and human skin, exhibiting a solid adhesion. In this process of adhesion for skin, the adhesive force is generated through the formation of two kinds of bonds. One is the electrostatic interaction formed by borax pentahydrate and the collagens with abundant charged sites in the tissue; the other is the dynamic covalent bonds facilitated by TA in the adhesive layer and the amino or thiol groups in the tissue (FIG. 5B). To evaluate the adhesive ability of the substance, the adhesive strength was quantified by the tensile adhesive test. As shown in FIG. 5B, the substrate material exhibits the greatest adhesion strength of 7.91 kPa to paper while also offering a desired adhesive strength of 2.83 kPa to pigskin. The results allow substrate material to adhere firmly to the skin of the human body when monitoring human motion without any fixation.

As shown in FIG. 5D, the substrate material can conduct the circuit again and light up the LED light after self-healing. The self-healing process of the substrate material is elucidated in FIG. 5E, highlighting the underlying mechanisms. This process relies on two types of dynamic bonds. Firstly, the healing is facilitated by the formation of dynamic catechol-borate bonds involving TA, enabling the reconstruction of the crosslinked network. Secondly, supramolecular interactions among PVA, glycerol, and TA molecules contribute to reversible bonds that are crucial for the healing processes. To investigate the self-repairing electrical performance of the substrate material, it was connected to a real-time current measuring system for further analysis. FIG. 5F clearly demonstrates that the current almost disappears when E-slime is completely severed. However, when the separated parts of the substrate material are reconnected, the circuit immediately restores its original current value. This observation indicates the rapid reconstruction of the 3D conductive network among the contacted interfaces of the healed hydrogel. Remarkably, the substrate material exhibits the ability to recover its original conductivity after severance in 10 consecutive cut-healing cycles. Additionally, the relationship between separate distance and self-healing time was studied (FIG. 5G). The substrate material has a fast self-healing time (less than 15 s) when the separate distance is less than 2 mm, and it can still heal itself even when the distance increases to 5 mm. The results confirm that the substrate material has a strong self-healing ability, enhancing its potential to resist external interference and damage in epidermal electronic applications.

Then, the influence of self-healing on the conductivity of E-slime is further investigated. As depicted in FIG. 5H, the conductivity of E-slime will stabilize at its initial value (˜0.32 S/m) after ten minutes, indicating that the electrical performance of the sensor remains unchanged before and after self-healing. Furthermore, the cut-healing cycle experiments were conducted to assess the robustness of the mechanical properties of the substrate material. As illustrated in FIG. 5I, the elastic modulus of the substrate material is almost unchanged (˜5.5 kPa) even after 100 cut-healing cycles, verifying a robust mechanical property. At the same time, FIG. 5J illustrates that the substrate material's resistance variation was less than 5% during the first 30 self-healing cycles. Even after 100 self-healing cycles, the relative resistance variation of the sensor remained below 7.5%. These results indicate that the sensor also possesses stable electrical self-healing performance. These findings validate the robust self-healing capabilities of the sensor, as well as the sustained stability of its electromechanical performance even after multiple self-healing cycles. This combination of electrical conductivity, self-recovery capability, and dynamic shaping ability positions E-slime as an ideal conductive material for in-situ soft electronics, particularly for applications in human motion detection.

### Applications of Substrate Material on Human Hand Motion Detection

Due to its appropriate electromechanical properties, plasticity, adhesion, and self-healing capabilities, the substrate material holds significant potential as an excellent strain sensor. Moreover, its reusability and customizability enable it to be easily and repeatedly attached to various locations on the human body. Herein, the hand motion sensing ability of the substrate material as a strain sensor was explored. FIG. 6A displays an overview of mounting one piece of the substrate material on various hand locations in different shapes to accommodate the corresponding motions, including the index finger (the first and second phalanges), thumb, thenar, and metacarpophalangeal joint (MCP joint).

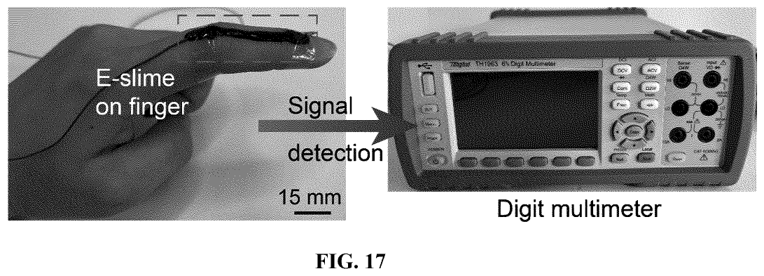

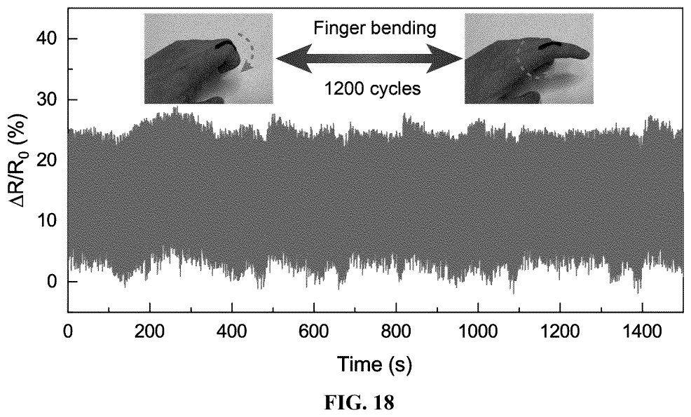

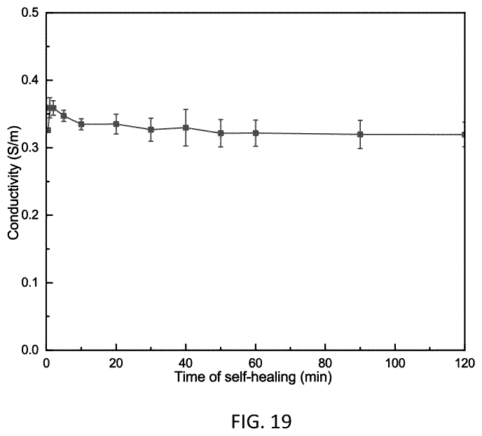

The substrate material was directly attached to the finger (the second phalange) surface without any adhesive to monitor the dynamic movement of this joint angle (experimental setup in FIG. 17). As shown in FIG. 6B, when the stretching angle of the finger increased from 60° to 120°, the relative resistance variation signal exhibited a correspondingly near-linear (R²=0.98) decrease (sensitivity ˜0.17%/°) due to the change of the substrate material strain, indicating that the substrate material has a high sensitivity. FIG. 6C illustrates the electrical signal response of the substrate material upon repeated measurements of finger bending motion at different angles (30°, 60°, and 120°). The results indicate that the substrate material has a rapid response rate to finger bending motion (˜2 s) and the effectiveness of repeated motion detection; the results regarding cyclic detection (1200 cycles in ˜1500 s) of finger motion further verify the durability of the substrate material (FIG. 18). As illustrated in FIG. 6D, when the substrate material was mounted in hand thenar, clear and stable signals of relative resistance variation could be detected as the generation of close and open (two angles) motion. To further investigate the application of the substrate material for entire hand motion detection, it was adhered to the MCP joint. As illustrated in FIG. 6E, each gesture, including “zero,” “one,” “two,” “three,” “four,” and “five,” produced a distinct resistance value that could be fully recovered.

In addition to the application for hand motion detection, the substrate material is also capable of monitoring the subtle movement of the hand due to its sensing abilities of motion speed and direction. By deploying E-slime onto the index finger, as demonstrated in FIG. 6F, the signal of relative resistance variation (˜2.5%) could be captured clearly when clicking the mouse each time. Although handwriting detection is challenging due to the involvement of unique characteristics (force, speed, and sequence of the writing), the substrate material can produce a complex but stable waveform for even a simple signature, as shown in FIGS. 6G-H. The results demonstrate that the substrate material can detect complex linguistic writing, e.g. not only English words but also Chinese characters, manifesting the substrate materials' potential in the application of real-time handwriting recognition. Besides, a substrate material sensing apparatus or system can be integrated through multiple pieces of substrate material units. As illustrated in FIGS. 6I-J, the substrate material sensing system (5 sensing units) was able to detect five finger motion signals simultaneously, including the cycles of the clenching fist (5 cycles) and hand gestures (5 types). The experimental results validate the application of E-slime in hand motion posture detection, further highlighting its potential as a flexible sensor or epidermal sensing system that can be formed in situ on the hand. This emphasizes its potential in applications for human-computer interfaces and gesture pattern recognition.

### Applications of Substate Material on Human Body Motion Detection

Beneficial from the convenient customizability, full reusability, strong skin adhesion, stable self-healing capabilities, and adaptability to the skin, the substrate material can be applied to various areas of the human body for detecting motion signals by in-situ formation of epidermal electronics, including joint movements and physiological micro-motions (FIG. 7A). As demonstrated in FIG. 7B, the substrate material detects facial protrusions and captures signals during frowning (relative resistance variation ˜−3%) and expresses amazement (relative resistance variation ˜7%) when attaching to the forehead. Additionally, when the substrate material is adequately adhered to the side of the mouth, the signals caused by smiling (relative resistance variation ˜12%), laughing (relative resistance variation ˜2%), and even big laughter (relative resistance variation ˜5%) can be consistently and clearly identified (FIG. 7c), demonstrating the potential of the substrate material in facial expression recognition applications.

Besides facial motion detection, the substrate material can also be applied as a flexible wearable sensor for body joint motion detection. As illustrated in FIG. 7D, the substrate material could accurately measure the electrical signals when the wrist is bent by 45° multiple times in both clockwise (relative resistance variation ˜−25%) and counterclockwise (relative resistance variation ˜10%) directions. When the elbow was bent multiple times at angles of 30°, 45°, and 60°, the substrate material was capable of measuring the corresponding relative resistance changes (˜11%, ˜19%, and 25%) clearly and consistently (FIG. 7E). Furthermore, as shown in FIG. 7F, the substrate material could also continuously detect the signal changes during multiple knee flexion and extension movements at different angles, including 30° flexion (relative resistance variation ˜2%), 30° extension (relative resistance variation ˜−10%), and 60° extension (relative resistance variation ˜−15%). As demonstrated in FIG. 7G, when altering the shape and size of the substrate material and attaching it to the ankle joint, it could accurately detect the electrical signal changes during joint extension (relative resistance variation −15%) and flexion (relative resistance variation ˜20%). The above results indicate that changing the deployment position and shape dimensions of the substrate material can accurately and distinctly detect the electrical signals of different body movements in a targeted manner. This further demonstrates its potential for human-computer interaction applications.

Moreover, the human body surface manifests delicate mechanical signals, including strain in the throat area triggered by vocalizations and strain in pulse locations. Due to the substrate material's exceptional sensitivity and ease of customization (in terms of shape and thickness), it can detect these faint signals. As demonstrated in FIG. 7H, the substrate material was specifically tailored into a thin rectangular film form and applied to the skin in the throat region, exhibiting a high sensitivity in detecting the corresponding electrical signals when swallowing (relative resistance variation ˜15%) or coughing (relative resistance variation ˜8%) occurs. Additionally, when the substrate material is customized into a circular disc shape and placed on the wrist at the pulse location, it can consistently and accurately detect the pulse (˜72 bpm) of the minute strains caused by pulse signals (FIG. 7I). In conclusion, the results confirm that E-slime exhibits excellent sensitivity, electromechanical stability, robust bio-adhesion, reliable self-healing capability, and the added advantages of reusability and easy customization. It can accurately detect both extensive human body motions (large deformations) and subtle changes in epidermal motion. As a result, the substrate material is a promising wearable strain sensor with flexible properties, offering significant potential for applications in human-computer interaction, facial expression recognition, and health monitoring.

### Experimental Section

Materials: Polyvinyl alcohol (PVA) with a molecular weight of 89000˜98000, tannic acid (TA) and glycerol (gly) with analytical reagent, borax pentahydrate with a 100 mesh, and graphite powder with a 10000 mesh were bought from Shanghai Macklin Biochemical Technology Co., Ltd. Carbon black powder with 200 mesh was supplied by Lion Specialty Chemicals Co., Ltd. All chemicals, solvents, and other consumable materials were purchased and used as received unless specially noted.

Preparation of E-slime: Under constant stirring with a water bath at 80° C. for 2 hours, 0.35 g PVA, 0.1 g glycerol, and 0.075 g TA were added and dispersed into 10 g deionized water. Then, 0.25 g graphite and 0.1 g carbon black were dissolved into the mixture and stirred for 10 min for complete dissolution under a 60° C. water bath. After stirring, 4% borax pentahydrate solution was added to the mixture drop by drop under stirring for polymerization. Finally, the electronic slime was obtained after 10 minutes of stirring.

Skin Replica Fabrication: Except for directly measuring the human electrical signal, pigskin purchased from a grocery store and cut into small pieces is utilized as a surrogate for human skin in all the experiments conducted in this paper. To emulate human skin, the pigskin undergoes degreasing and hair removal.

Tensile Tests of E-slime: The tensile test was evaluated on a tensimeter (LT-5000, Reotai Precision Instrument Co., Ltd) equipped with a 10 N load cell at the crosshead speed of 60 mm/min. The tested cylindrical samples were 20 mm long and with a gauge length of 5 mm.

Adhesion Performance Tests of Substrate Material: The adhesion strength tests were conducted on the tensimeter (LT-5000, Reotai Precision Instrument Co., Ltd). The tested sample was stuck to two pieces of substrates with a bonding area of 10×10 mm². After the sample bonded together, the adhered substrates were conducted to a lap shear stress when applied to the aforementioned tensimeter with a crosshead speed of 10 mm/min. By calculating the maximum force divided by the initial adhered area, the maximum stress was obtained as the adhesive strength.

Reusability Tests of Substrate Material: A testing sample of Substrate Material changed shape from circular to rectangular, which was repeated as a process for each cycle. For each reusing cycle, the elastic modulus, conductivity, and adhesive strength were evaluated.

Self-healing Performance Tests of Substrate Material: A testing sample of E-slime was cut into two pieces with a distance, which was repeated as a process for each cycle. For each self-healing cycle, the elastic modulus and conductivity were tested.

Electrical Tests of Substrate Material: The substrate material was prepared into a cubic shape with a size of 20 mm length, 10 mm width, and 2 mm thickness for the electrical performance tests. By using a digital multimeter (TH1963A, Tonghui Electronics Co., Ltd), the electrical signals of E-slime were obtained. The conductivity (σ) of the substate material testing sample was calculated by the formula σ=L/(R×A), where L is the length of the testing sample, R is the resistance of the sample, and A represents the cross-sectional area of the sample. As for the relative resistance variation, it was determined by the equation ΔR/R₀=(R−R₀)/R₀, where R₀ and R are the resistance without and with applied strain, respectively.

Experimental Setup of E-slime on Motion Detection: The substrate material was applied to the targeted location of the human body in different shapes and sizes. By using pressure-sensitive tape (Kefu Medical Technology Co., Ltd), the wires were tightly fixed onto the E-slime. The digital multimeter (TH1963A, Tonghui Electronics Co., Ltd) could detect electrical signals in real time.

Cell Viability Tests of Substrate Material: C2C12 myoblasts were purchased from ATCC and maintained in the standard CO₂ incubator (Thermo Scientific Heracel 150i) with the environment as 5% CO₂ and 37° C., while cells were cultured in the growth medium (GM) of Dulbecco's Modified Eagle's Medium as supplemented with 10% Fetal Bovine Serum and 1% Penicillin-Streptomycin-Neomycin (Gibco). Samples of hydrogels, including PVA, PVA/gly, PVA/gly/TA, and E-slime, were washed with 75% ethanol and exposed under UV light for sterilization prior to experiments. Each of the samples (2 g) was immersed in GM for 2 hours; after removing the samples, the remaining solutions were used to test for biocompatibility. C2C12 cells were first seeded in Petri dishes at a density of 3.0×10⁴ cells/cm² and cultured in GM for 24 h. Then, GM was substituted with different remaining solutions for culture cells for another 2 hours. In addition, the cells continued to grow in GM as a control. After that, a LIVE/DEAD Cell Imaging kit containing Calcein AM and Propidium Iodide (Life Technologies Co., Ltd) was used to calculate the cell viability.

### EXAMPLES

Studies leading to the present invention have shown that the workable contents of the ingredients in the substrate material may vary in certain ranges. Table 4 below provides a summary of the workable wt % ratio of some of the ingredients in the substrate material. Table 5 below provides a summary of workable wt % ratio of graphite and carbon black in the substrate material. Table 6 corresponds to Table 4 but is presented to illustrate the workable wt % ratio of the ingredients including water in the substrate material.

### Conclusion

Epidermal electronics play a significant role in healthcare and human-machine interfaces due to their ability to provide biological signal monitoring, detect body movements, and sense subtle motions through custom designs tailored to individual body shapes and sizes. However, widespread utilization of epidermal electronics encounters barriers related to complex production methods and high material costs. Developing epidermal electronics that achieve appropriate electrical and mechanical performance, adhesive properties, and self-healing capability remains an ongoing challenge. The present invention provides an ultra-deformable, bio-adhesive, self-healing, and electromechanical-durable wearable electronic substrate material for epidermal electronics applications. The substrate material E-slime is based on a biocompatible PVA/gly/TA hydrogel formulation, and it can be easily fabricated via a simple water bath process using raw materials that are readily available, low-cost, and environmentally friendly. Through the introduction of conductive carbon-based dopants of graphite and carbon black, the substrate material achieves the desired electrical, mechanical, and physiological sensing properties for flexible electronics. Furthermore, the substrate material also exhibits high conformability, adhesiveness, and self-healing attributes that allow it to seamlessly interface with skin and withstand external mechanical interferences. In addition, it can be reused multiple times through simple peeling-off and reapplication processes. Thus, the substrate material can be applied to motion detection across diverse human body locations in this work, and the experimental results demonstrate the versatility of the substrate material, which is integrable in applications such as personal health tracking and human-machine interfacing.

### Tables

TABLE 1
Comparison in the electromechanical parameters, reusable cycles, and self-healing ability
between this work and other Silly Putty-like materials in literature reports.

| | | | | | | | | | | Maximum | Fastest | |
|---|---|---|---|---|---|---|---|---|---|---|---|---|
| | | | | Sensing | Maximum | Detection | Adhesive | | | self- | self- | |
| | Gauge | | | range | stretch | limit | strength | | Reusable | healing | healing | |
| Reference | factor | Conductivity | | (strain) | distance | (strain) | (skin) | | cycles | distance | time | |
| E-slime | 2.95 | 0.33 | S/m | 400% | 2600% | 1% | 3 | kPa | 100 | 5 mm | 1 | s |
| ^{[1]} | 0.4 | N/A | | 200% | 1200% | N/A | N/A | | N/A | N/A | 0.15 | s |
| ^{[2]} | 8.21 | 0.1 | S/m | 500% | 1500% | N/A | N/A | | N/A | N/A | N/A | |
| ^{[3]} | 1.31 | 0.025 | S/m | 400% | 2000% | N/A | 5 | kPa | N/A | N/A | 0.5 | s |
| ^{[4]} | N/A | 0.08 | S/m | N/A | N/A | N/A | 6.5 | kPa | N/A | N/A | N/A | |
| ^{[5]} | 1.23 | 1.44 | S/m | N/A | 80% | N/A | 3.9 | kPa | N/A | N/A | N/A | |
| ^{[6]} | 1.15 | 0.32 | S/m | 650% | 1000% | N/A | 1 | kPa | N/A | N/A | 5 | s |
| ^{[7]} | 2.56 | N/A | | 250% | 1085% | N/A | N/A | | N/A | N/A | 30 | s |
| ^{[8]} | 1 | 0.6 | S/m | 400% | 4300% | N/A | 5.25 | kPa | N/A | N/A | 10 | s |
| ^{[9]} | 2 | 0.05 | S/m | N/A | N/A | N/A | 4.5 | kPa | N/A | N/A | 4 | s |

| ^{[10]} | 0.8 | 0.03 | S/m | 150% | 1160% | N/A | N/A | N/A | N/A | N/A | |
|---|---|---|---|---|---|---|---|---|---|---|---|
| ^{[11]} | N/A | 0.01 | S/m | 150% | 570% | N/A | N/A | N/A | N/A | 5 | s |

TABLE 2
Comparison in the complexity of fabrication and
required equipment among this work and other Silly
Putty-like materials in literature reports.

| Refer- | Substrate | Dopant | | |
|---|---|---|---|---|
| ence | material | materials | Crosslinker | Equipment |
| E-slime | PVA, glycerol, | Graphite, | Borax | Heating |
| | TA | carbon black | pentahydrate | |
| [1] | PVA | MXene | Sodium | Heating |
| | | | tetraborate | |
| [2] | PAAm, CNF, | MXene | Borax | Freeze, UV |
| | TA | | | light |
| [3] | α-Lipoic acid, | Iron (III) | N/A | Heating |
| | Sodium | nitrate | | |
| | alginate | nonahydrate | | |
| [4] | SBMA, PEGDA | PEDOT:PSS | N/A | Vacuum pump |
| [5] | GelMA | CNT | K₂S₂O₈ | Heating, |
| | | | | freeze, UV |
| | | | | light |
| [6] | B. mori silk, | Lithium | Sodium | Heating, dry, |
| | PVA | bromide | tetraborate | centrifuge |
| [7] | PVA | Microcrys- | Borax | Heating, freeze |
| | | talline | | |
| | | cellulose | | |
| [8] | PVA | MXene, PPy | Sodium | Ball milling, |
| | | | borate | heating |
| [9] | PVA, PDA | CNT | borate | Heating |
| [10] | Acrylic acid | N/A | Pluronic | Heating, UV |
| | | | F127 | light |
| | | | diacrylate | |
| [11] | PVA, chitosan | CNT | Boric acid | Heating |

TABLE 3
Prices of common epidermal electronic system
materials (CNY per gram/milliliter)

| | Material | | | | | |
|---|---|---|---|---|---|---|
| | Silver nanowires | MXene | CNT | Graphite | Tannic acid | Carbon black |
| Price | 4600 | 2500 | 2300 | 1926 | 3.5 | 2.98 |

| | Material | | | | | |
|---|---|---|---|---|---|---|
| | Sodium alginate | Chitosan | PVA | Gelatin | Graphite | Glycerol |
| Price | 1.76 | 1.44 | 1.28 | 0.79 | 0.18 | 0.1 |

Data from source: Shanghai Macklin Reagent Co., Ltd.

The cost (unit-price) of all the chemicals that used to manufacture E-slime can be calculated as follows:

$$Cost = \frac{3.5 \times 0.05 + 2.98 \times 0.1 + 1.28 \times 0.35 + 0.18 \times 0.25 + 0.1 \times 0.1}{11} = 0.089 yuan / g$$

TABLE 4
Workable wt % ratio of some of ingredients substrate material

| Example | PVA (wt %) | Glycerol (wt %) | TA (wt %) | Water (wt %) |
|---|---|---|---|---|
| 1 | 0.35 | 0.0625 | 0 | 10 |
| 2 | 0.35 | 0.0625 | 0.025 | 10 |
| 3 | 0.35 | 0.0625 | 0.05 | 10 |
| 4 | 0.35 | 0.0625 | 0.1 | 10 |
| 5 | 0.35 | 0.0625 | 0.15 | 10 |
| 6 | 0.35 | 0.125 | 0 | 10 |
| 7 | 0.35 | 0.125 | 0.025 | 10 |
| 8 | 0.35 | 0.125 | 0.05 | 10 |
| 9 | 0.35 | 0.125 | 0.1 | 10 |
| 10 | 0.35 | 0.125 | 0.15 | 10 |
| 11 | 0.35 | 0.175 | 0 | 10 |
| 12 | 0.35 | 0.175 | 0.025 | 10 |
| 13 | 0.35 | 0.175 | 0.05 | 10 |
| 14 | 0.35 | 0.175 | 0.1 | 10 |
| 15 | 0.35 | 0.175 | 0.15 | 10 |
| 16 | 0.35 | 0.225 | 0 | 10 |
| 17 | 0.35 | 0.225 | 0.025 | 10 |
| 18 | 0.35 | 0.225 | 0.05 | 10 |
| 19 | 0.35 | 0.225 | 0.1 | 10 |
| 20 | 0.35 | 0.225 | 0.15 | 10 |

Table 5 below provides a summary of workable wt % ratio of graphite and carbon black in the substrate material.

| | Graphite | Carbon Black | |
|---|---|---|---|
| Ratio | (g) | (g) | Conductivity Notes |
| 1:1 | 0.10 | 0.10 | Lower conductivity |
| 1.5:1 | 0.15 | 0.10 | Increased conductivity |
| 2:1 | 0.20 | 0.10 | Higher conductivity |
| 2.5:1 | 0.25 | 0.10 | Maximum conductivity |
| 3:1 | 0.30 | 0.10 | Decreasing conductivity |
| 3.5:1 | 0.35 | 0.10 | Further decreased |
| | | | conductivity |
| 4:1 | 0.40 | 0.10 | Lowest conductivity |

Table 6 illustrates the workable wt % ratio of the ingredients including water in the substrate material.

| | PVA | Glycerol | TA | Water |
|---|---|---|---|---|
| Example | (wt %) | (wt %) | (wt %) | (wt %) |
| 1 | 3.41 | 0.24 | 0 | 96.35 |
| 2 | 3.41 | 0.24 | 0.10 | 96.25 |
| 3 | 3.41 | 0.24 | 0.24 | 96.11 |
| 4 | 3.41 | 0.24 | 0.48 | 95.87 |
| 5 | 3.41 | 0.24 | 0.71 | 95.64 |
| 6 | 3.39 | 0.48 | 0 | 96.13 |
| 7 | 3.39 | 0.48 | 0.10 | 96.03 |
| 8 | 3.39 | 0.48 | 0.24 | 95.89 |
| 9 | 3.39 | 0.48 | 0.48 | 95.65 |
| 10 | 3.39 | 0.48 | 0.71 | 95.42 |
| 11 | 3.37 | 0.72 | 0 | 95.91 |
| 12 | 3.37 | 0.72 | 0.10 | 95.81 |
| 13 | 3.37 | 0.72 | 0.24 | 95.67 |
| 14 | 3.37 | 0.72 | 0.48 | 95.43 |
| 15 | 3.37 | 0.72 | 0.71 | 95.20 |
| 16 | 3.34 | 0.95 | 0 | 95.71 |
| 17 | 3.34 | 0.95 | 0.10 | 95.61 |
| 18 | 3.34 | 0.95 | 0.24 | 95.47 |
| 19 | 3.34 | 0.95 | 0.48 | 95.23 |
| 20 | 3.34 | 0.95 | 0.71 | 95.00 |

### REFERENCES

The following references are incorporated in their entirety and a skilled person is considered to be aware of disclosure of these references.
M. Khatib, O. Zohar, H. Haick, Advanced Materials 2021, 33, 2004190.
Z. Rao, F. Ershad, A. Almasri, L. Gonzalez, X. Wu, C. Yu, Adv Materials Technologies 2020, 5, 2000233.
H. Lim, H. S. Kim, R. Qazi, Y. Kwon, J. Jeong, W. Yeo, Advanced Materials 2020, 32, 1901924.
C. Xu, S. A. Solomon, W. Gao, Nat Mach Intell 2023, 5, 1344.
M. Qi, R. Yang, Z. Wang, Y. Liu, Q. Zhang, B. He, K. Li, Q. Yang, L. Wei, C. Pan, M. Chen, Adv Funct Materials 2023, 33, 2214479.
D. Gao, J. Lv, P. S. Lee, Advanced Materials 2022, 34, 2105020.
J. Chen, Y. Zhu, X. Chang, D. Pan, G. Song, Z. Guo, N. Naik, Adv Funct Materials 2021, 31, 2104686.
Z. Li, Y. Li, Z. Wang, P. Wu, N. Liu, K. Liu, Z. Gu, Y. Chen, J. Nie, H. Shao, Y. He, Nano Energy 2023, 108, 108166.
B. Nie, S. Liu, Q. Qu, Y. Zhang, M. Zhao, J. Liu, Acta Biomaterialia 2022, 139, 280.
S. Zhang, C. Liu, X. Sun, W. Huang, Progress in Materials Science 2022, 128, 100962.
D.-H. Kim, N. Lu, R. Ma, Y.-S. Kim, R.-H. Kim, S. Wang, J. Wu, S. M. Won, H. Tao, A. Islam, K. J. Yu, T. Kim, R. Chowdhury, M. Ying, L. Xu, M. Li, H.-J. Chung, H. Keum, M. McCormick, P. Liu, Y.-W. Zhang, F. G. Omenetto, Y. Huang, T. Coleman, J. A. Rogers, Science 2011, 333, 838.
Y. Ma, Y. Zhang, S. Cai, Z. Han, X. Liu, F. Wang, Y. Cao, Z. Wang, H. Li, Y. Chen, X. Feng, Advanced Materials 2020, 32, 1902062.
Y. Lee, J. Park, A. Choe, S. Cho, J. Kim, H. Ko, Adv Funct Materials 2020, 30, 1904523.
Y. Luo, M. R. Abidian, J.-H. Ahn, D. Akinwande, A. M. Andrews, M. Antonietti, Z. Bao, M. Berggren, C. A. Berkey, C. J. Bettinger, J. Chen, P. Chen, W. Cheng, X. Cheng, S.-J. Choi, A. Chortos, C. Dagdeviren, R. H. Dauskardt, C. Di, M. D. Dickey, X. Duan, A. Facchetti, Z. Fan, Y. Fang, J. Feng, X. Feng, H. Gao, W. Gao, X. Gong, C. F. Guo, X. Guo, M. C. Hartel, Z. He, J. S. Ho, Y. Hu, Q. Huang, Y. Huang, F. Huo, M. M. Hussain, A. Javey, U. Jeong, C. Jiang, X. Jiang, J. Kang, D. Karnaushenko, A. Khademhosseini, D.-H. Kim, I.-D. Kim, D. Kireev, L. Kong, C. Lee, N.-E. Lee, P. S. Lee, T.-W. Lee, F. Li, J. Li, C. Liang, C. T. Lim, Y. Lin, D. J. Lipomi, J. Liu, K. Liu, N. Liu, R. Liu, Y. Liu, Y. Liu, Z. Liu, Z. Liu, X. J. Loh, N. Lu, Z. Lv, S. Magdassi, G. G. Malliaras, N. Matsuhisa, A. Nathan, S. Niu, J. Pan, C. Pang, Q. Pei, H. Peng, D. Qi, H. Ren, J. A. Rogers, A. Rowe, O. G. Schmidt, T. Sekitani, D.-G. Seo, G. Shen, X. Sheng, Q. Shi, T. Someya, Y. Song, E. Stavrinidou, M. Su, X. Sun, K. Takei, X.-M. Tao, B. C. K. Tee, A. V.-Y. Thean, T. Q. Trung, C. Wan, H. Wang, J. Wang, M. Wang, S. Wang, T. Wang, Z. L. Wang, P. S. Weiss, H. Wen, S. Xu, T. Xu, H. Yan, X. Yan, H. Yang, L. Yang, S. Yang, L. Yin, C. Yu, G. Yu, J. Yu, S.-H. Yu, X. Yu, E. Zamburg, H. Zhang, X. Zhang, X. Zhang, X. Zhang, Y. Zhang, Y. Zhang, S. Zhao, X. Zhao, Y. Zheng, Y.-Q. Zheng, Z. Zheng, T. Zhou, B. Zhu, M. Zhu, R. Zhu, Y. Zhu, Y. Zhu, G. Zou, X. Chen, ACS Nano 2023, 17, 5211.
Y. Guo, X. Wei, S. Gao, W. Yue, Y. Li, G. Shen, Adv Funct Materials 2021, 31, 2104288.
J. Luo, J. Yang, X. Zheng, X. Ke, Y. Chen, H. Tan, J. Li, Adv Healthcare Materials 2020, 9, 1901423.
W. B. Han, J. H. Lee, J. Shin, S. Hwang, Advanced Materials 2020, 32, 2002211.
M. Asulin, I. Michael, A. Shapira, T. Dvir, Advanced Science 2021, 8, 2004205.
T. La, S. Qiu, D. K. Scott, R. Bakhtiari, J. W. P. Kuziek, K. E. Mathewson, J. Rieger, H. Chung, Adv Healthcare Materials 2018, 7, 1801033.
D. Kireev, S. K. Ameri, A. Nederveld, J. Kampfe, H. Jang, N. Lu, D. Akinwande, Nat Protoc 2021, 16, 2395.
N. Gogurla, Y. Kim, S. Cho, J. Kim, S. Kim, Advanced Materials 2021, 33, 2008308.
L. Tang, J. Shang, X. Jiang, Sci. Adv. 2021, 7, eabe3778.
N. Gogurla, S. Kim, Advanced Energy Materials 2021, 11, 2100801.
C.-Y. Yang, M.-A. Stoeckel, T.-P. Ruoko, H.-Y. Wu, X. Liu, N. B. Kolhe, Z. Wu, Y. Puttisong, C. Musumeci, M. Massetti, H. Sun, K. Xu, D. Tu, W. M. Chen, H. Y. Woo, M. Fahlman, S. A. Jenekhe, M. Berggren, S. Fabiano, Nat Commun 2021, 12, 2354.
G.-H. Lee, H. Woo, C. Yoon, C. Yang, J.-Y. Bae, W. Kim, D. H. Lee, H. Kang, S. Han, S.-K. Kang, S. Park, H.-R. Kim, J.-W. Jeong, S. Park, Advanced Materials 2022, 34, 2204159.
H. Huang, Y. Feng, X. Yang, Y. Shen, Biosensors and Bioelectronics 2022, 214, 114547.
M. Li, A. Pal, J. Byun, G. Gardi, M. Sitti, Advanced Materials 2023, 35, 2304825.
M. Sun, C. Tian, L. Mao, X. Meng, X. Shen, B. Hao, X. Wang, H. Xie, L. Zhang, Advanced Functional Materials 2022, 32, 2112508.
D. Suh, K. P. Faseela, W. Kim, C. Park, J. G. Lim, S. Seo, M. K. Kim, H. Moon, S. Baik, Nat Commun 2020, 11, 2252.
S. Zhang, B. Xu, X. Lu, L. Wang, Y. Li, N. Ma, H. Wei, X. Zhang, G. Wang, J. Mater. Chem. C 2020, 8, 6763.
Y.-Z. Zhang, K. H. Lee, D. H. Anjum, R. Sougrat, Q. Jiang, H. Kim, H. N. Alshareef, Science Advances 2018, 4, eaat0098.
M. Chen, B. B. Murphy, Y. Wang, F. Vitale, S. Yang, Small 2023, 19, 2205854.
L. Hu, P. L. Chee, S. Sugiarto, Y. Yu, C. Shi, R. Yan, Z. Yao, X. Shi, J. Zhi, D. Kai, H. Yu, W. Huang, Advanced Materials 2023, 35, 2205326.
Z. Wang, H. Zhou, J. Lai, B. Yan, H. Liu, X. Jin, A. Ma, G. Zhang, W. Zhao, W. Chen, J. Mater. Chem. C 2018, 6, 9200.
H. Zheng, N. Lin, Y. He, B. Zuo, ACS Appl. Mater. Interfaces 2021, 13, 40013.
M. Liao, P. Wan, J. Wen, M. Gong, X. Wu, Y. Wang, R. Shi, L. Zhang, Adv Funct Materials 2017, 27, 1703852.
Z. Wang, Y. Cong, J. Fu, J. Mater. Chem. B 2020, 8, 3437.
X. Huang, C. Chen, X. Ma, T. Zhu, W. Ma, Q. Jin, R. Du, Y. Cai, M. Zhang, D. Kong, M. Wang, J. Ren, Q. Zhang, X. Jia, Adv Funct Materials 2023, 33, 2302846.
T. Ke, L. Zhao, X. Fan, H. Gu, Journal of Materials Science & Technology 2023, 135, 199.
Y. Wei, L. Xiang, H. Ou, F. Li, Y. Zhang, Y. Qian, L. Hao, J. Diao, M. Zhang, P. Zhu, Y. Liu, Y. Kuang, G. Chen, Advanced Functional Materials 2020, 30, 2005135.
J. Zhang, L. Wan, Y. Gao, X. Fang, T. Lu, L. Pan, F. Xuan, Advanced Electronic Materials 2019, 5, 1900285.
Y. Fan, X. Wang, X. Zhang, Z. Chang, W. Kuang, H. Tian, ACS Appl. Polym. Mater. 2023, 5, 8430.
Z. Qin, G. Zhao, Y. Zhang, Z. Gu, Y. Tang, J. T. Aladejana, J. Ren, Y. Jiang, Z. Guo, X. Peng, X. Zhang, B. B. Xu, T. Chen, Small2023, 19, 2303038.
F. Lin, Z. Wang, Y. Shen, L. Tang, P. Zhang, Y. Wang, Y. Chen, B. Huang, B. Lu, Journal of Materials Chemistry A 2019, 7, 26442.
J. Seo, S. Oh, G. Choi, H. Kim, J. Kim, T. Hwang, Y. Mun, C. Kim, J. Choi, S. H. Kim, E. Lee, H. S. Lee, ACS Appl. Polym. Mater. 2022, 4, 6495.
L. Zhao, Z. Ren, X. Liu, Q. Ling, Z. Li, H. Gu, ACS Appl. Mater. Interfaces 2021, 13, 11344.
M. Song, H. Yu, J. Zhu, Z. Ouyang, S. Y. H. Abdalkarim, K. C. Tam, Y. Li, Chemical Engineering Journal 2020, 398, 125547.
Y. Zhong, P. Li, X. Wang, J. Hao, Green Chemistry 2021, 23, 2496.
W. Zhang, Y. Deng, J. Zhao, T. Zhang, X. Zhang, W. Song, L. Wang, T. Li, Small 2023, 19, 2207360.
C. Xie, X. Wang, H. He, Y. Ding, X. Lu, Advanced Functional Materials 2020, 30, 1909954.

What is claimed is:

1. A substrate material comprising a matrix ingredient of polyvinyl alcohol (PVA), a plasticizing agent of glycerol (gly), and a conductive material of graphite carbon black, and water.

2. A substrate material, as claimed in claim 1, further comprising a physical property-enhancing agent of tannic acid (TA).

3. A substrate material, as claimed in claim 2, wherein the physical property enhancing agent is adapted to provide crosslinking, adhesion, and/or flexibility in the substrate material.

4. A substrate material as claimed in claim 3, comprising a polymerization agent for improving cross-linking in the substrate material.

5. A substrate material as claimed in claim 1, comprising 3.34-3.41 wt % of the polyvinyl alcohol (PVA) and 0.24-0.95 wt % of the glycerol (gly).

6. A substrate material as claimed in claim 5, wherein the substrate material has 1.865 to 3.22 wt % of the conductive material.

7. A substrate material as claimed in claim 6, comprising a polymerization agent for improving cross-linking in the substrate material, wherein the polymerization agent is selected from a group including borax pentahydrate, borax decahydrate and glutaraldehyde, and wherein the substrate material preferably has 1-6 wt % of the polymerization agent and more preferably 1-4 wt % of the polymerization agent.

8. A substrate material as claimed in claim 7, comprising up to 0.71 wt % of the tannic acid (TA).

9. A substrate material as claimed in claim 6, wherein the ratio of graphite to carbon black in the conductive material is from 1:1 to 2.5:1.

10. A substrate material as claimed in claim 2, wherein the wt ratio of the polyvinyl alcohol (PVA), glycerol (gly), tannic acid, and conductive material is 0.35:0.0625˜0.225:0˜0.15.

11. A substrate material as claimed in claim 8, wherein the substrate material has an adhesive strength of 2.42 kPa to 5.84 kPa, an elastic modulus of 2.67 kPa to 11.72 kPa, and a stretchability of up to 2600%.

12. A substrate material, as claimed in claim 1, wherein the substrate material assumes the formation of a layer or a strip for attachment or engagement to the skin of a subject.

13. An electronic apparatus comprising a substrate material of claim 1, further comprising an electronic device for recording electrical signals received through the substrate material, wherein the electrical signals are indicative of one or more physiological or physical parameters of a subject on which the electronic apparatus is applied or one or more parameters of the surroundings to the subject.

14. An electronic apparatus as claimed in claim 13, wherein the parameters are selected from the group including blood glucose level of the subject, electrocardiogram (ECG) of the subject, facial expression of the subject, temperature of the surroundings, humidity of the surroundings, motion and/or posture of the subject, wound healing status of the subject, and pressure or change of pressure at a certain location of the subject.

15. A method of making of a substrate material, comprising the steps of:
adding a predetermined amount of a matrix ingredient of polyvinyl alcohol (PVA) and a plasticizing agent of glycerol (gly) to deionized water to form a first solution, and mixing the solution for a period of time under a predetermined temperature, and
adding a conductive material of graphite and carbon black to the first solution forming a second solution, and mixing the second solution for a period of time under a predetermined temperature to form a third solution.

16. A method as claimed in claim 15, comprising a step of also adding tannic acid (TA) to the first solution before adding the conductive material.

17. A method as claimed in claim 16, comprising a step of adding a polymerization agent in the third solution, wherein the polymerization agent is selected from a group including borax pentahydrate and borax decahydrate or glutaraldehyde.

18. A method as claimed in claim 16, wherein the first solution is stirred for 1-4 hours under 70-90° C. before adding the conductive material, and wherein the second solution is stirred for 5-20 mins under 70-90° C. before allowing the second solution to take form.

19. A method as claimed in claim 18, comprising a step of shaping the formed substrate material into a predetermined configuration.

20. A method as claimed in claim 16, wherein the substrate material has 3.34-3.41 wt % of the polyvinyl alcohol (PVA), 0.24-0.95 wt % of the glycerol (gly), 1.865 to 3.22 wt % of the conductive material, and up to 0.71 wt % of the tannic acid (TA), and the concentration of the polymerization agent is 1-6%.
